(19) United States
(12) Patent Application Publication
(10) Pub. No.: US 2026/0367099 A1
(43) Pub. Date: Nov. 19, 2026

(54) MULTIPLEX ABO ANTIBODY ASSAY AND METHOD

(71) Applicant: The Governors of the University of Alberta, Edmonton (CA)

(72) Inventors: Lori West, Edmonton (CA); Anne M. Halpin, Edmonton (CA); Todd Lambert Lowary, Edmonton (CA); Christopher Cairo, Edmonton (CA); Bruce Motyka, Edmonton (CA)

(21) Appl. No.: 19/130,182

(22) PCT Filed: Nov. 15, 2023
(86) PCT No.: PCT/CA2023/051539

§ 371 Date: May 15, 2025

Related U.S. Application Data
(60) Provisional application No. 63/425,637, filed on Nov. 15, 2022.

(51) Int. Cl. G01N 33/80; G01N 21/64; G01N 33/58

(52) U.S. Cl. CPC G01N 33/80; G01N 21/6428; G01N 33/582; G01N 2021/6439

(57) ABSTRACT

The present application provides a multiplex assay for identifying an ABO histo-blood subtype antibody profile of a subject, comprising incubating a biological sample from the subject with a bead composition that contains Luminex® beads individually coupled to Type I, Type II, Type III, Type IV ABO-A and ABO-B subtype glycan antigens. The antigen-coupled beads each comprise a bovine serum albumin (BSA)-linked ABO subtype antigen coupled to the corresponding beads at an amount of about 5 μg of the BSA-linked ABO subtype antigen per 1.0×10⁶ beads. Also provided are compositions and kits for performing the multiplex assay.

### CROSS REFERENCE TO RELATED APPLICATION

This patent application claims the benefit of priority to U.S. Provisional Patent Application No. 63/425,637, filed Nov. 15, 2022, and entitled, “Multiplex ABO Antibody Assay and Method”, which is incorporated in its entirety herein by this reference.

### FIELD OF THE INVENTION

The present application pertains to the field of ABO blood group antibody detection. More particularly, the present application relates to an assay and method for detecting and quantifying anti-ABO antigen subtype antibodies, for example, in the fields of organ and cell transplantation, and blood transfusion. The method and assay employ a glycan assay to enable simultaneous detection and precise assessment of antibodies towards multiple ABO antigen subtype structures. This technology can also be used to detect glycan structures relevant in xenotransplantation.

### INTRODUCTION

It has been over a century since Karl Landsteiner first described the ABO blood groups (Landsteiner 2001). Additional knowledge of A, B, and H antigen structures (H antigen defining blood group O) has since been gained, but detection of ABO antibodies in clinical practice and related research is still reliant on variations of the erythrocyte agglutination method described in 1901. Hemagglutination is known to be plagued by a lack of sensitivity and poor reproducibility. (Datta et al. 2021; Denomme and Anani 2020; Kang, Lim, and Baik 2014) It is also cumbersome to distinguish between IgG and IgM isotypes by agglutination and it is not possible to define ABH glycan-subtype specificities. Yet ABO hemagglutination titres are routinely used to inform clinical management of transfusion and ABO-incompatible (ABOi) transplantation.

Knowledge of ABH glycobiology has evolved including the sequencing of the genes that encode for the glycosyltransferases that decorate human cells and tissues with these carbohydrate structures (Lane 2016; de Mattos 2016; Oriol et al. 1992; Pendu et al. 1989). It is known that there are six glycan structure subtypes (I-VI) of each A, B, and H antigens, as denoted in Table 1.

The six subtypes of each of the three major ABH antigens are denoted in Table 1. Table 1: ABO histo-blood group antigen subtypes.

TABLE 1
ABH histo-blood group antigen subtypes

| Type | Carbohydrate |
|---|---|
| Type I | ABO-β-(1→3)-β-D-Glcp NAc |
| Type II | ABO-β-(1→4)-β-D-Glcp NAc |
| Type III | ABO-β-(1→3)-α-D-Galp NAc |
| Type IV | ABO-β-(1→3)-β-D-Galp NAc |
| Type V | ABO-β-(1→3)-β-D-Galp |
| Type VI | ABO-β-(1→4)-β-D-Glcp |

The A, B and H subtype glycans are not equally represented on various cells and tissues. The most biologically relevant antigens are reported to be A-II, III, IV and B-II. (Bentall et al. 2021; Clausen and Hakomori 1989; Jeyakanthan et al. 2016; de Mattos 2016). In the case of ABO-A individuals, assuming the more common A1 subgroup, subtype A-II is the only A antigen found on vascular endothelium, but erythrocytes and (some) epithelial cells are additionally decorated with A-III and A-IV glycans. Tissues of ABO-B individuals appear to have only B-II glycans although not all tissues have been well studied; this work is hampered by the limited availability of B subtype-specific monoclonal antibodies. (Clausen et al. 1985; Jeyakanthan et al. 2015; Ravn and Dabelsteen 2000)

ABO A2 is a common subgroup of ABO-A blood groups. Approximately 20% of all ABO-A individuals are ABO-A2 rather than the most common A1. ABO-A2 individuals have only ABO-A-II structures on all cells and tissues. (Svensson et al. 2009) For this reason ABO-A2 kidney donors are used for transplant into ABO-O and ABO-B recipients who have disproportionally long wait times as compared to ABO-A and ABO-AB recipients. This increasing clinical practice to use A2 donors calls for new technology that can specifically detect antibodies to the A-II glycan structures and exclude antibodies to the clinically irrelevant A-III and A-IV antigen structures.

Some glycobiology studies use A- and B-trisaccharides as surrogates for A- and B-subtype glycans, however, since these surrogate glycans are not biologically relevant, anti-A- and B-trisaccharide-specific antibodies are of uncertain significance. (Pochechueva et al. 2011; Stussi et al. 2005)

Naturally occurring antibodies to non-self ABH glycans present a major immunologic barrier in transplantation and transfusion. As the hemagglutination assay does not distinguish glycan subtype-specificities of ABO antibodies, it remains challenging to fully characterise antibody profiles, resulting in inaccurate risk assessment. The subtype-specificity is critical to assessment of ABO antibodies in ABOi transplantation due to tissue-specific glycan representation on endothelial and epithelial cells and erythrocytes. (Bentall et al. 2021; Jeyakanthan et al. 2016) An ABH-glycan microarray has previously been developed to improve precision and accuracy of ABO antibody detection (WO 2013/029181). This method was not implemented in clinical laboratories, however, due in part to lack of readily available instruments, lack of expertise in this method in the clinical laboratory setting, and lack of proper optimization. (Bentall et al. 2021; Daga et al. 2021; Jeyakanthan et al. 2015, 2016) Furthermore, using this previous method, it was a challenge to achieve intra- and inter-laboratory standardization. (Jeyakanthan et al. 2016; Muthana and Gildersleeve 2016).

Luminex® methods and technologies are now widely used in clinical and research laboratory antibody detection assays. Individual polystyrene beads (also referred to interchangeably as microspheres or microbeads) are coupled with target antigens; up to 500 beads can be distinguished from one another by their different hue intensities. Following incubation of patient serum (or plasma) with antigen-coupled beads, phycoerythrin (PE)-labelled secondary antibodies are used to detect serum antibodies bound to each individual bead, with the Luminex® instrument reporting PE fluorescence intensity of each bead. The instrument settings are automatically determined in its calibration, making it a highly reproducible method within and between clinical laboratories. Indeed, this rapid, more sophisticated antibody detection technique has become the clinical standard for histocompatibility laboratories worldwide in the detection of antibodies to human leukocyte antigens (HLA) to support solid organ and hematopoietic stem cell transplantation (HSCT) (El-Awar, Lee, and Terasaki 2005; Sullivan, Gebel, and Bray 2017; Tait et al. 2013).

The presence of Luminex® instruments and expertise in HLA-histocompatibility laboratories makes this highly standardized method a natural fit for ABO-histocompatibility antibody profiling. However, difficulties with, for example, suitable loading and reproducible and stable coupling of ABH-glycans has meant that the use of this method for ABO-histocompatibility antibody detection has not been successfully implemented.

The above information is provided for the purpose of making known information believed by the applicants to be of possible relevance to the present invention. No admission is necessarily intended, nor should be construed, that any of the preceding information constitutes prior art against the present invention.

### SUMMARY OF THE INVENTION

An object of the present application is to provide a multiplex ABO antibody assay and method that are useful for precise and comprehensive ABO antibody detection and characterization. One purpose of the present assay system and method is to provide a specific and sensitive means for detection, immune risk assessment, and pre- and post-transplant management of ABO-related compatibility between donors and recipients in the clinical setting of transplantation and similar aspects of transfusion. The present method and system provide improved accuracy and ABO glycan specificity in donor and recipient compatibility assessment pre-transplant and in planning intentional ABO-incompatible transplants with the greatest degree of safety. The present method and system are further useful in monitoring a patient's antibodies post-transplant, to promptly identify early stages of rejection, thus enabling rapid clinical intervention to prevent or minimize damage from antibody-mediated injury. The present method and assay system can also facilitate improved matching between recipient and donor blood products in the case of transfusion. Identification of unexpected transfusion reactions and atypical ABO blood group antibodies would reduce the number of cases where the incorrect blood product is provided to an individual. The present method can also be used to screen whole blood donors; whole blood donation can be used in the setting of trauma injury and requires the use of ABO-O donors who have been shown to have low levels of anti-A and anti-B antibodies.

In accordance with an aspect of the present application, there is provided method of identifying an ABO histo-blood subtype antibody profile of a subject comprising:
(a) incubating a biological sample obtained from said subject with a bead composition, wherein the bead composition comprises:
   (i) a plurality of subsets of Luminex® beads where each of the subsets within the plurality are individually coupled to a Type I, Type II, Type III, Type IV ABO-A or ABO-B subtype glycan antigen;
   (ii) a first control subset of Luminex® beads that are coupled to a negative control antigen (e.g., BSA) and function as a negative control;
   (iii) a second control subset of Luminex® beads that are not coupled to any antigen and function as a further negative control; and
   (iv) a third control subset Luminex® bead that are coupled to a positive control antigen (e.g., galactose-α-1,3-galactose) and function as a positive control;
(b) washing the incubated mixture from step (a);
(c) incubating the washed mixture from step (b) with secondary anti-IgG and anti-IgM antibodies, wherein the secondary antibodies are labeled with a fluorescent label;
(d) washing the incubated mixture from step (c) to remove unbound secondary antibodies;
(e) measuring fluorescence associated with the subsets of Luminex® beads; and
(f) generating the ABO antigen subtype antibody profile of the subject from the fluorescence data collected in step (e),

wherein the subsets of beads coupled to the ABO-A and ABO-B subtype glycans each comprise a bovine serum albumin (BSA)-linked ABO subtype antigen coupled to the corresponding beads at an amount of about 5 μg of the BSA-linked ABO subtype antigen per 1.0×10⁶ beads.

In accordance with one embodiment, there is provided a kit for identifying an ABO histo-blood subtype antibody profile of a subject comprising:
(a) a bead composition, wherein the bead composition comprises:
   (i) a plurality of subsets of Luminex® beads where each of the subsets within the plurality are individually coupled to a Type I, Type II, Type III, Type IV ABO-A or ABO-B subtype glycan antigen;
   (ii) a first control subset of Luminex® beads that are coupled to a negative control antigen (e.g., BSA) and function as a negative control;
   (iii) a second control subset of Luminex® beads that are not coupled to any antigen and function as a further negative control; and
   (iv) a third control subset Luminex® beads that are coupled to a positive control antigen (e.g., galactose-α-1,3-galactose) and function as a positive control; and
(b) instructions for use of the kit to perform the method described above.

In some embodiments, the bead composition, for use in the above method or as a component of the above-defined kit, additionally comprises two subsets of Luminex® beads individually coupled to BSA-linked Type V ABO-A and ABO-B subtype glycan antigens and/or the bead composition additionally comprises two subsets of Luminex® beads individually coupled to BSA-linked Type VI ABO-A and ABO-B subtype glycan antigens and/or the bead composition additionally comprises subsets of Luminex® beads individually coupled to BSA-linked Type Type I, Type II, Type III, Type IV, Type V and/or Type VI ABO-H glycan antigens.

In some embodiments, the bead composition, for use in the above method or as a component of the above-defined kit, additionally comprises one or more subsets of Luminex® beads individually coupled to BSA-linked antigen(s) present in a donor animal for a xenotransplant, wherein the antigen(s) present in the donor animal is, optionally, N-Glycolylneuraminic acid (Neu5Gc), N-acetylneuraminic acid (Neu5Ac), or both.

### BRIEF DESCRIPTION OF TABLES AND FIGURES

For a better understanding of the application as described herein, as well as other aspects and further features thereof, reference is made to the following description which is to be used in conjunction with the accompanying drawings, where:

FIG. 1 schematically depicts an overview of the ABO antibody detection assay of the present invention and an example of the results from one ABO-A individual. Individual Luminex® beads were coupled to each ABO-A and ABO-B glycan subtype antigen. Negative control beads included a bead coupled to BSA as well as a bead that was not coupled with any target. A positive control bead was coupled to α-Gal glycan. Serum or plasma was added to duplicate wells for the addition of either IgG and IgM secondary antibody. This example of an ABO antibody profile was from an ABO-A1/O genotyped individual with a hemagglutination titre against ABO-B erythrocytes of 1/32 and a negative hemagglutination titre against ABO-A1 erythrocytes.

FIG. 2 graphically depicts titration results for selection of the required glycan-antigen coupling concentration (μg of BSA-linked antigen per ml of bead suspension containing 1.0×10⁶ beads/ml);

FIG. 3 is an example of a graphical confirmation that the A and B glycan subtype coupling was highly consistent for the beads;

FIG. 4 graphically depicts a comparison between the present multiplex assay and a previous ABH-glycan microarray assay on glass slides, which illustrates that the Luminex® assay was able to detect antibodies with specificities to ABO-A subtype glycans with more sensitivity than the microarray assay; this increased sensitivity was observed most clearly at the lower concentrations of antibody;

FIG. 5 illustrates assay results from 24 individual runs of a positive control serum showing excellent reproducibility of the multiplex assay described herein (these results are shown as the mean±/− the 95% confidence interval);

FIG. 6 graphically depicts a comparison of IgG and IgM antibodies by blood group, showing that there was a wide range of IgG and IgM anti-ABO antibodies in healthy adult individuals across the ABO-O, -A, and -B blood groups. ABO-O individuals (n=68) produced higher levels of IgG anti-A than ABO-B individuals (n=17), and higher IgG anti-B compared to ABO-A individuals (n=48). In contrast, IgM isotype anti-A and anti-B antibodies did not vary based on the ABO blood group. This figure also highlights the differences in anti-A-II, III, and IV IgG antibody levels observed in ABO-O individuals;

FIG. 7 graphically depicts assay results using sera from ABO-O individuals showing that there were no significant differences in the level of ABO antibodies between females (n=47) and males (n=21) (anti-A is shown as an average of A-II, III, and IV subtype-specificities and anti-B is only B-II subtype-specificity);

FIG. 8 illustrates a comparison of antibodies with specificities to A and B trisaccharides to antibodies specific for the cell- and tissue-relevant tetrasaccharide glycan subtypes A-II, -III, and -IV and B-II. This comparison shows that it is common for ABO-A individuals to have high levels of IgG and IgM anti-A-trisaccharide antibodies (i.e., biologically irrelevant antibodies with reactivity to “self” A), together with relatively low antibody levels to A-II, III, and IV subtype glycans. In contrast, ABO-B individuals are unlikely to have either anti-B-trisaccharide or anti-B-II antibodies;

FIG. 9 graphically depicts the results of an assay of healthy sera showing no differences between IgG and IgM anti-α-Gal antibody levels (n=132). There were also no differences by sex (n=83 females and n=49 males);

FIG. 10 graphically depicts the results of sera tested in parallel by red cell agglutination and analysis using the multiplex assay (n=119), showing a wide range of IgG and IgM antibody levels within each hemagglutination antibody titre;

FIG. 11 graphically depicts a comparison of IgG and IgM antibody levels to hemagglutination titres in only the ABO-O individuals (n=54), demonstrating the same variability as shown in FIG. 6;

FIG. 12 graphically illustrates that the pan-reactive anti-H antibodies in Bombay phenotype subjects is readily visualized using the multiplex assay according to one embodiment of the present application, further facilitating definition of antibody isotype (IgG and IgM) patterns in these subjects; and

FIG. 13 graphically depicts the results of an assay of renal transplant candidates assessed for ABO-A incompatible transplantation (n=88), illustrating a wide distribution of anti-H antibodies among these candidates.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “ABO antigen subtype,” refers to glycan subtypes of the A, B and H (O) antigens. The ABO histo-blood group antigens are glycan structures, which are always found as part of a larger glycan structure, and the adjacent residues allow the carbohydrate antigens to be classed into subtypes. Each of these ABO antigens is present as one of six subtypes denoted in Table 1 above. It is possible that additional subtypes will be identified in the future. The present assay and method can be adapted to take advantage of information relating to the presence or absence, or the amount, of such yet to be identified subtypes.

As used herein, the term “anti-ABO antigen subtype antibody profile,” refers to the antibodies present in a sample or a subject with specificities to ABO histo-blood group antigen subtypes. A basic profile includes information as to the presence or absence of at least one of the anti-ABO antigen subtype antibodies; a full profile can further include information as to the absolute or relative amounts of the antibodies present in a sample or a subject. The profile can further include information as to the isotypes of the anti-ABO antigen subtype antibodies present. In one example, the anti-ABO antigen subtype antibody profile includes information as to the presence or absence, or as to the absolute or relative amounts, and/or isotypes, of anti-ABO antigen subtype antibodies specific for more than one ABO histo-blood group antigen subtype, such as more than one of the 18 major ABO histo-blood group antigen subtypes. In another example, the anti-ABO antigen subtype antibody profile includes information as to the presence or absence, or as to the absolute or relative amounts, of anti-ABO antigen subtype antibodies specific for the 18 major ABO histo-blood group antigen subtypes.

As used herein, the term “ABO subgroup,” is used to refer to a subgroup within the broad ABO blood group classification. These subgroups can be characterized by qualitative and quantitative differences in ABO antigen profiles.

As used herein, the term “biological sample,” refers to a blood or tissue-derived sample that contains ABO subtype antigens or anti-ABO antigen subtype antibodies, which includes, but is not limited to: organ, tissue, and erythrocytes [ABO subtype antigens] and serum, plasma, and blood (i.e., whole blood) and other blood products (e.g., IVIG), and monoclonal antibodies [anti-ABO antigen subtype antibodies]. The biological sample can be a patient sample or a healthy individual considered as a control.

Abbreviations used herein include:

| | |
|---|---|
| ABOi | ABO-incompatible |
| AHG | Anti-human globulin |
| BSA | Bovine serum albumin |
| α-Gal | Galactose-α-1,3-galactose |
| DTT | Dithiothreitol |
| HLA | Human leukocyte antigen |
| HSCT | Hematopoietic stem cell transplant |
| MFI | Mean fluorescence intensity |
| PE | Phycoerythrin |
| IVIG | Intravenous IgG immunoglobulin |

The present inventors have developed a multiplex assay to characterize A, B and H glycan subtype-specific antibodies in a patient or control sample, for example a human serum sample, or a monoclonal antibody, and define antibody isotypes. In some embodiments the multiplex assay is a bead-based assay in which a plurality of subsets of beads are employed, with each subset of beads having a unique associated detection label (e.g., a unique embedded fluorescence). In some embodiments, the multiplex assay is a Luminex®-based assay, as illustrated herein as an example of such a bead-based assay.

The present multiplex assay overcomes many of the limitations associated with the current clinical hemagglutination assay typically used to evaluate A and B antibody levels as hemagglutination cannot determine glycan subtype-specific antibodies in patient samples. The present method and system have been optimized to facilitate reproducible and reliable results from patient samples (or monoclonal antibody preparations) and across different testing sites (e.g., clinical or research labs). This multiplex assay is useful for, for example, ABH-histocompatibility assessment for organ and cell transplantation and transfusion medicine, and for research in the field of glycoimmunology.

The method and multiplex assay described herein provides a means for physicians and other clinicians to assess ABO-compatibility or—incompatibility accurately and, consequently, to allow transplantation or transfusion boundaries to be safely established. Use of present method and system can reduce adverse reactions resulting from unintentional or accidental use of incompatible blood products and organs/tissues/cells, and will assist in allowing intentional incompatibilities to be used safely, thus saving lives.

The present method and multiplex assay system address the major limitation of hemagglutination, that A, B, and H glycan subtypes are differentially expressed on erythrocytes and in various tissues. This has important implications for both transplantation and transfusion matching. In one embodiment, the present method comprises the use of a device having multiple ABO histo-blood group subtype antigens coupled to a carrier, for example, a glycan micro- or macro-array. The device is then used to measure antibodies towards the ABO antigen subtypes qualitatively or quantitatively. This method is useful, for example, because it can allow for degrees of transplantation and transfusion compatibility to be assessed more accurately than current available methods allow.

In accordance with one aspect there is provided an immunological method for determining the anti-ABO antigen subtype antibody profile of a subject. The method comprises the step of determining the presence or absence of antibodies against ABH antigen subtypes within a biological sample obtained from a subject. Typically, the biological sample is blood serum or plasma (but can also be from blood products or monoclonal antibody preparations.

The present method comprises obtaining a biological sample from a subject having A, B, and/or H glycan subtype antibodies, incubating the sample with a plurality of multiplexed A, B, and H antigen subtypes, and detecting binding complexes formed by the binding of the plurality of multiplexed A, B, and H antigen subtypes with said subtype antibodies present in the sample. The bound antibodies are then detected in such a manner that the identity of each anti-A, B, or H subtype-specific antibodies can be measured. The level of each subtype antibody is also determined by the level of fluorescence intensity output, for example, using a Luminex® instrument.

The multiplex assay employs a composition comprising a combination of subsets of beads, each subset being coupled with a different A, B, or H subtype antigen, thereby allowing simultaneous detection and measurement of the A, B, or H subtype-specific antibodies in the sample. In a particular embodiment, the present assay makes use of Luminex® beads, available from Luminex.

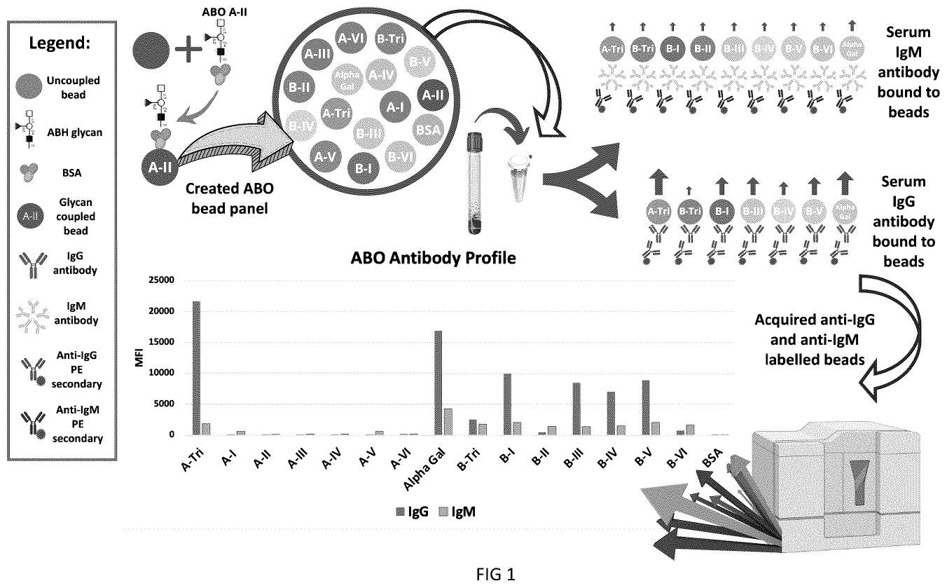

FIG. 1 provides a broad overview of the ABO antibody detection multiplex assay of the present application and an example of results obtained from an ABO-A individual. Briefly, in this example, the assay employs individual A- and B-subtype glycans coupled to individual subsets of beads, or beads; H glycans can also be included although not shown in this example. The method comprises incubating the glycan-coupled beads with a biological sample, such as a plasma or serum sample from a patient, washing the mixture to remove unbound antibodies and then detecting the presence of antibodies in the sample that have bound to the glycans on the beads. The bound antibodies are detected using secondary detection antibodies having a fluorescent label (e.g., phycoerythrin). The mean fluorescence intensity (MFI) for each subset of beads is detected as a measure of the ABO antibodies present in the biological sample, for example using Luminex® instrumentation.

In some embodiments, the secondary antibodies can be anti-human antibodies (i.e., one that will recognize and bind to human antibody) or it can be an antibody that has specificity for any human antibody of a specific isotype, which can be, but is not limited to, anti-human IgG (e.g., IgG1, IgG2, IgG3, IgG4), IgM, IgE, IgD and/or IgA antibodies or any combination of these antibodies.

In specific embodiments the method employs both IgG- and IgM-specific secondary antibodies for detection. Optionally, the method additionally employs IgA-specific secondary antibodies for detection.

The present inventors have found that IgG and IgM ABO-A and B antibodies vary widely within each ABO-A and -B titre as detected by hemagglutination. The relative amounts of these antibodies within a detected antibody profile provide additional information useful in characterising compatibility or incompatibility between a donor and recipient (for transplantation or transfusion).

In determining the anti-A, B, or H subtype-specific antibody profile of the subject, it is advantageous to test for the presence or absence of antibodies against most or all of the ABO antigen subtypes. In accordance with one embodiment, the composition comprising a combination of subsets of beads includes subsets of beads individually coupled to Type I, Type II, Type III, Type IV ABO-A or ABO-B subtype glycan antigens, respectively. Since there is a degree of cross-reactivity of antibodies for Type V subtype antigens with Type Ill and IV subtype antigens, it is not always necessary to include beads coupled to the Type V subtype antigens. Similarly, since there is a degree of cross-reactivity of antibodies for Type VI subtype antigens with Type II subtype antigens, it is not always necessary to include beads coupled to the Type VI subtype antigens. However, in some embodiments, the composition comprising a combination of subsets of beads includes subsets of beads individually coupled to Type I, Type II, Type III, Type IV, Type V and Type VI ABO-A or ABO-B subtype glycan antigens, respectively. Inclusion of subtypes I through VI can allow for definition of clinically relevant antibody profiles.

In some embodiments, the composition of antigen-coupled beads additionally comprises one or more bead subset, each coupled to an ABO-H Type I, Type II, Type III, Type IV, Type V or Type VI subtype glycan antigen. Use of such a composition in the multiplex assay described herein can be useful to characterise the A, B and H antibody profile of a subject. This can be particularly beneficial, for example, for identifying subjects with the Bombay phenotype, who lack the normal fucosyltransferase 1 (FUT1) gene. In order to avoid complications during transplantation or transfusion, it is important to detect Bombay phenotype individuals. However, the standard tests for ABO blood group system (e.g., hemagglutination) would incorrectly identify these individuals as having O type blood. Since anti-H immunoglobulins can activate the complement cascade, it will lead to the lysis of red blood cells while they are still in the circulation, provoking an acute hemolytic reaction. This can be avoided by typing subjects to identify the Bombay phenotype using a composition and assay according to the present application.

In an alternative embodiment, the multiplex assay described herein can be adapted for use in recipient risk assessment for xenotransplantation. In this embodiment, the composition of antigen-coupled beads contains subsets of beads that are individually coupled to ABO-H and/or xeno-glycan antigens represented in the donor animal/organ. In some embodiments, the composition comprises a subset of beads coupled to a non-human sialic acid. In some non-limiting embodiments, the composition of antigen-coupled beads comprises a subset of beads coupled to N-glycolylneuraminic acid (Neu5Gc) and a subset of beads coupled to N-acetylneuraminic acid (Neu5Ac). Neu5Gc and Neu5Ac are sialic acid molecules synthesized by the gene cytidine monophospho-N-acetylneuraminic acid hydroxylase (CMAH).

Each antigen-coupled bead composition additionally comprises a control subset of beads coupled to a positive control antigen and another control subset of beads coupled to a negative control antigen.

In a particular embodiment, the positive control antigen is the galactose-α-1,3-galactose (α-Gal) epitope (α-Gal-(1→3)-β-Gal-(1→ (3) 4)-GlcNAc-R), which is found on glycolipids and glycoproteins of non-primate mammals and New World monkeys. All humans are known to produce antibodies specific for α-Gal. By applying the present optimized coupling technique to this antigen on a subset of the beads, it is possible to confirm that the assay is functioning properly by noting the presence of this positive control signal indicating the formation of binding complexes on the designated bead address in the panel. Further, incorporation of this antigen can also be useful to provide surrogate for normal levels of antibody in the sample.

Negative control beads are essential to this assay to ensure that non-specific reactivity to the platform itself is not affecting the assay output. In this assay these beads are designed based on known potential targets for assay non-specificity as has been done in histocompatibility assays. In one example, the negative control antigen is bovine serum albumin (BSA). BSA is also used in preparing the glycan-protein conjugate for coupling of the glycan antigens to the beads. Accordingly, use of BSA as the negative control is useful to confirm that antibodies in the sample are binding to the glycan antigens rather than to the protein used in the conjugate. A secondary negative control bead is not coupled to any antigen and is used to assess for non-specific reactivity to the bead surface. These controls are essential for clinical assays and required to meet clinical laboratory accreditation standards as defined by accrediting bodies.

### Coupling of Glycan Antigens to Beads

As noted above, A, B, and H antigens are glycans. The synthesis of the glycans with the prerequisite alkene aglycone has been previously reported. (Jeyakanthan et al. 2015, 2016; Meloncelli and Lowary 2009, 2010; Meloncelli, West, and Lowary 2011) The glycan antigens are coupled to the beads glycan-protein conjugates. To prepare the glycan-protein conjugates, the glycan subtype antigens were synthesized according to the previously described synthetic methods and BSA was linked to amines in the glycan antigen structures.

In accordance with some embodiments, there is provided a bead composition comprising a plurality of subsets of Luminex® beads where each of the subsets within the plurality are individually coupled to a Type I, Type II, Type III, Type IV, Type V, and/or Type VI ABO-A or ABO-B subtype glycan antigen.

In a particular example, the composition comprises the following BSA-linked Type I, Type II, Type III, Type IV ABO-A or ABO-B subtype glycan antigens coupled to respective subsets of Luminex® beads:
A Type I α-D-GalpNAc-(1→3)-[α-L-Fucp-(1→2)]-β-D-Galp-(1→3)-β-D-GlcpNAc-BSA,
A Type II α-D-GalpNAc-(1→3)-[α-L-Fucp-(1→2)]-β-D-Galp-(1→4)-β-D-GlcpNAc-BSA,
A Type Ill α-D-GalpNAc-(1→3)-[α-L-Fucp-(1→2)]-β-D-Galp-(1→3)-α-D-GalpNAc-BSA,
A Type IV α-D-GalpNAc-(1→3)-[α-L-Fucp-(1→2)]-β-D-Galp-(1→3)-β-D-GalpNAc-BSA,
B Type I α-D-Galp-(1→3)-[α-L-Fucp-(1→2)]-β-D-Galp-(1→3)-β-D-GlcpNAc-BSA,
B Type II α-D-Galp-(1→3)-[α-L-Fucp-(1→2)]-β-D-Galp-(1→4)-β-D-GlcpNAc-BSA,
B Type Ill α-D-Galp-(1→3)-[α-L-Fucp-(1→2)]-β-D-Galp-(1→3)-α-D-GalpNAc-BSA, and
B Type IV α-D-Galp-(1→3)-[α-L-Fucp-(1→2)]-β-D-Galp-(1→3)-β-D-GalpNAc-BSA

In other embodiments, the bead composition comprises subsets of Luminex® beads coupled to BSA-linked Type V ABO-A and ABO-B subtype glycan antigens. In some examples, the BSA-linked Type V ABO-A and ABO-B subtype glycan antigens are:
A Type V α-D-GalpNAc-(1→3)-[α-L-Fucp-(1→2)]-β-D-Galp-(1→3)-β-D-Galp-BSA, and
B Type V α-D-Galp-(1→3)-[α-L-Fucp-(1→2)]-β-D-Galp-(1→3)-β-D-Galp-BSA.

In other embodiments, the bead composition comprises subsets of Luminex® beads coupled to BSA-linked Type VI ABO-A and ABO-B subtype glycan antigens (with or without beads coupled to BSA-linked Type V ABO-A and ABO-B subtype glycan antigens). In some examples, the BSA-linked Type VI ABO-A and ABO-B subtype glycan antigens are:
A Type VI α-D-GalpNAc-(1→3)-[α-L-Fucp-(1→2)]-β-D-Galp-(1→4)-β-D-Glcp-BSA, and
B Type VI α-D-Galp-(1→3)-[α-L-Fucp-(1→2)]-β-D-Galp-(1→4)-β-D-Glcp-BSA.

In other embodiments, the bead composition comprises subsets of Luminex® beads individually coupled to BSA-linked Type I, Type II, Type III, Type IV, Type V and Type VI ABO-H subtype glycan antigens. In some examples, the BSA-linked Type I, Type II, Type III, Type IV, Type V and Type VI ABO-H subtype glycan antigens are:

Additional beads/beads are coupled to galactose-α-1,3-galactose (α-Gal) and to BSA as positive and negative control beads, respectively. Blood group A- and β-trisaccharides can also be coupled to individual beads. For example, the Type VI ABO-A and ABO-B subtype glycan antigens are:
H Type I α-L-Fucp-(1→2)-β-D-Galp-(1→3)-β-D-GlcpNAc-BSA,
H Type II α-L-Fucp-(1→2)-β-D-Galp-(1→4)-β-D-GlcpNAc-BSA,
H Type III α-L-Fucp-(1→2)-β-D-Galp-(1→3)-α-D-GalpNAc-BSA,
H Type IV α-L-Fucp-(1→2)-β-D-Galp-(1→3)-β-D-GalpNAc-BSA,
H Type V α-L-Fucp-(1→2)-β-D-Galp-(1→3)-β-D-Galp-BSA, and
H Type VI α-L-Fucp-(1→2)]-β-D-Galp-(1→4)-β-D-Glcp-BSA.

The present inventors have found that in order to provide reproducible results using Luminex® beads, it was necessary to adjust the loading ratio of the glycan antigens on the beads to significantly higher than the standard loading ratio. In particular, using titration studies, the inventors have found that it is necessary to load approximately 5 μg of the BSA-linked ABO subtype antigen per 1.0×10⁶ beads, which is about five times higher than the amount recommended by the manufacturer. Typically, the use of higher loading values is avoided to minimize non-specific binding during the assay. However, without wishing to be bound by theory, this particular loading concentration may be required because of the unique structure and nature of the glycan-protein conjugates. As the ratio of protein to bead is different than a strict protein coupling protocol, the specific ratio of antigen to bead is essential. Insufficient levels of antigens on each bead decreases the sensitivity of the assay overall. Accurate transplant risk assessment demands high sensitivity of antibody detection platforms as well as consistent levels of antigen from bead to bead. The consistency of the antigen binding is demonstrated in FIG. 3.

### Use of the Multiplex Assay in ABO Matching for Transplantation

Improved matching in transplantation is achieved using the presently described method and multiplex assay by measuring a potential transplant recipient's antibodies towards the tissue-specific ABO histo-blood group glycan subtype antigens. This antibody profile information, combined with the knowledge of antigen subtype expression on the donor organ or tissue, is used to determine the degree of compatibility of such a transplant. The present multiplex assay makes it possible not only to measure antibodies towards all 18 ABO antigen subtypes as well as assay control targets but also to identify different antibody isotypes in a quantitative and reproducible manner.

Different cells and tissues express different A and B antigen subtypes with varying distribution. While it is known that A, B, and H antigen subtypes are expressed on various tissue surfaces, it has only recently been appreciated by clinicians that this subtype expression can vary between different tissues in the same individual. Simply considering the ABO blood type of a donor and assessing the level of anti-A and anti-B antibodies using (red cell) hemagglutination, as is currently practiced in evaluating suitability of a donor organ or tissue, completely disregards known variance in A and B glycan subtypes expressed on the different donor organs and tissues. The lack of glycan subtype-specific antibody information does not allow for accurate transplant immune risk assessment and may unnecessary exclude individuals from access to ABO-incompatible transplantation thus contributing to inequity in access to transplant.

In one embodiment of the present application, a potential recipient can be assessed for suitability for an ABO-incompatible organ or tissue transplant using the present multiplex assay. The relevant anti-ABO glycan subtype-specific antibody profile would be determined. The patient would be considered for ABOi transplantation in the context of the relevant tissue/organ-specific antigen subtypes. In the setting of low antibody levels towards the relevant ABO antigen subtypes present on the donor organ a transplant could be considered a low immunologic risk. This ability to determine subtype-specific antibodies is especially important in cases where eligibility for intentional ABO-incompatible transplantation may be denied due to detection of antibodies in a hemagglutination assay that are, in fact, irrelevant to the organ/tissue being transplanted.

Post-transplant, the patient could be further monitored for antibodies towards the donor A or B glycan subtypes present on the donor organ using the multiplex assay of the present application. Any development of antibodies or increased antibody quantity could be detected and appropriate clinical measures enacted. Confirmation of absent or low antibodies to the tissue target antigen(s), would likewise be helpful in avoiding unnecessary treatment interventions that may be triggered by detection of (irrelevant) antibodies in the hemagglutination assay.

In another embodiment, a potential recipient can be assessed for suitability for an ABO-compatible transplant using the present multiplex assay. In this case, the patient's serum antibodies are assessed to identify unexpected serum reactions potentially due to patient ABO subgroup that are less common. This assessment would be achieved using the present multiplex assay, identifying and characterizing any antibodies present towards all or subset of the 18 ABO antigen subtypes. It is important that there are only low antibody levels towards the ABO antigen subtypes present on the donor organ. In ABO-compatible transplantation, this analysis is less important than in the case of intentional ABO-incompatible transplantation. However, in rare cases unexpected antibody-mediated reactions do occur and are thought to contribute to chronic graft damage. Through the present method many of these reactions can be identified beforehand and any risks minimized. Ideally, serum from the organ donor should also be assessed for antibody levels towards the ABO antigen subtypes. This approach helps to identify unexpected ABO-incompatibilities, acting as a failsafe prior to transplantation.

In another embodiment, a potential recipient can be assessed for compatibility with a xenotransplantation of an organ from a non-human donor. In this case, the patient's serum antibodies are assessed to identify potential reactivity against the donor organ due to the presence of antibodies against one or more xeno-antigen found in the donor organ and/or expressed by the donor animal. This assessment would be achieved using the present multiplex assay, identifying and characterizing any antibodies present towards one or more xeno-antigen. Again, it is important there are no, or only low, antibody levels towards the one or more xeno-antigen.

### Use of the Multiplex Assay in ABO Matching in Transfusion

Improved matching in transfusion is achieved using the presently provided multiplex assay, by measuring serum antibodies of the potential recipient towards the ABO histo-blood group glycan subtypes present in the donor product prior to transfusion. This information can then be used to aid in the precise identification and etiology of unexpected transfusion reactions and assist in identifying the most compatible blood products. One potential application is detection of A and B antibodies in the setting of ABO-incompatible platelet transfusion which is a common clinical practice. In other cases, discrepancies are caused by rare ABO subgroups; therefore, the ability to quantify anti-A and B antibodies at the subtype and isotype level would be of value to clinical laboratorians and physicians. This will facilitate the selection of compatible blood products. As new A, B, and H glycan subtypes are identified, the present multiplex assay can be readily adaptable to account for newly discovered blood group antigen variants. Specifically, the bead composition can be adapted and expanded to incorporate additional antigen-coupled beads in which the antigen is the newly discovered variant.

To gain a better understanding of the invention described herein, the following examples are set forth. It should be understood that these examples are for illustrative purposes only. Therefore, they should not limit the scope of this invention in any way.

### EXAMPLES

### Example 1: Abo Subtype Antigen Coupling to Luminex® Beads

This example summarizes a specific procedure for ABO subtype antigen coupling to Luminex® beads, and optimization of the procedure conditions. The basic procedure is adapted from the August 2016 Rev E guidelines from Luminex's User Manual for the xMAP® Antibody Coupling Kit.

### Methods and Results

The coupling process was performed using a xMAP® 40-50016 Antibody Coupling Kit and Luminex® MagPlex® beads (e.g., MagPlex®-C Beads, Region 012) to couple BSA-linked ABO subtype antigens.

The ABO subtype antigens were A and B tetrasaccharide antigens (A-VI) that were synthesized and BSA-linked using methods previously developed in the Lowary Laboratory at the University of Alberta (Meloncelli P J et al 2010, Meloncelli P J et al 2011 and Meloncelli P J et al 2009). A detailed summary of the glycans used is found in Jeyakanthan et al. 2016. All antigens were reconstituted to 2 mg/ml in 14Ω water and stored at −80 °C until thawed for use.

| | | Coupled to |
|---|---|---|
| | | MagPlex ™ |
| Antigen | Source | Bead ID |
| A-I tetrasaccharide | Lowary Laboratory | 12 |
| A-II tetrasaccharide | Lowary Laboratory | 13 |
| A-III tetrasaccharide | Lowary Laboratory | 14 |
| A-IV tetrasaccharide | Lowary Laboratory | 15 |
| A-V tetrasaccharide | Lowary Laboratory | 18 |
| A-VI tetrasaccharide | Lowary Laboratory | 19 |
| B-I tetrasaccharide | Lowary Laboratory | 45 |
| B-II tetrasaccharide | Lowary Laboratory | 46 |
| B-III tetrasaccharide | Lowary Laboratory | 47 |
| B-IV tetrasaccharide | Lowary Laboratory | 48 |
| B-V tetrasaccharide | Lowary Laboratory | 51 |
| B-VI tetrasaccharide | Lowary Laboratory | 53 |
| BSA | Sigma Aldrich Cat#A3059 | 20 |
| α-Gal (Gala1-3Galb1- | Dexra UK Cat#NGP0334 | 21 |
| 4GlcNAc-BSA (3 atom | | |
| spacer) | | |
| No antigen (water) | Li Ka Shing Core Ultra | 22 |
| | Pure Water | |
| A-trisaccharide | Lowary Laboratory | 61 |
| B-trisaccharide | Lowary Laboratory | 62 |

### Coupling Procedure

Luminex MagPlex® Beads beads were coupled to individual ABO-A and ABO-B subtype glycans that were manufactured as previously described. (Jeyakanthan et al. 2015, 2016; Meloncelli and Lowary 2009, 2010; Meloncelli et al. 2011)

Briefly, A and B glycan subtype I-VI tetrasaccharide antigens were synthesized and bovine serum albumin (BSA) was linked to amines in these carbohydrate structures. The coupling procedure was performed as per the protein coupling procedure recommended by Luminex bead manufacturers (Angeloni et al. 2016). The ratio of antigen to bead was optimized for this glycan-protein antigen target using titration studies. The xMAP Antibody Coupling (AbC) Kit® was used for further standardization of the coupling procedure as detailed below.

### ABO-A and ABO-B Subtype Glycan Coupling to Beads

The required volume of Luminex® beads were resuspended and washed using the Activation buffer from the Coupling Kit. The beads were incubation with the Sulfo-NHS (N-hydroxysulfosuccinimide) reagent and freshly prepared EDC (1-ethyl-3-(3-dimethylaminopropyl) carbodiimide hydrochloride) reagent for 20 minutes at room temperature in the dark. The activated beads were washed using the Activation buffer to remove excess Sulfo-NHS and EDC. A BSA-linked ABO subtype antigen was added to the washed beads in Activation buffer, at an amount of 5 μg per 1.0×10⁶ beads, and incubated at room temperature in the dark for about 2 hours with mixing on a tube rotator. The ABO-subtype antigen coupled beads were washed using the Wash buffer from the Coupling Kit and then counted.

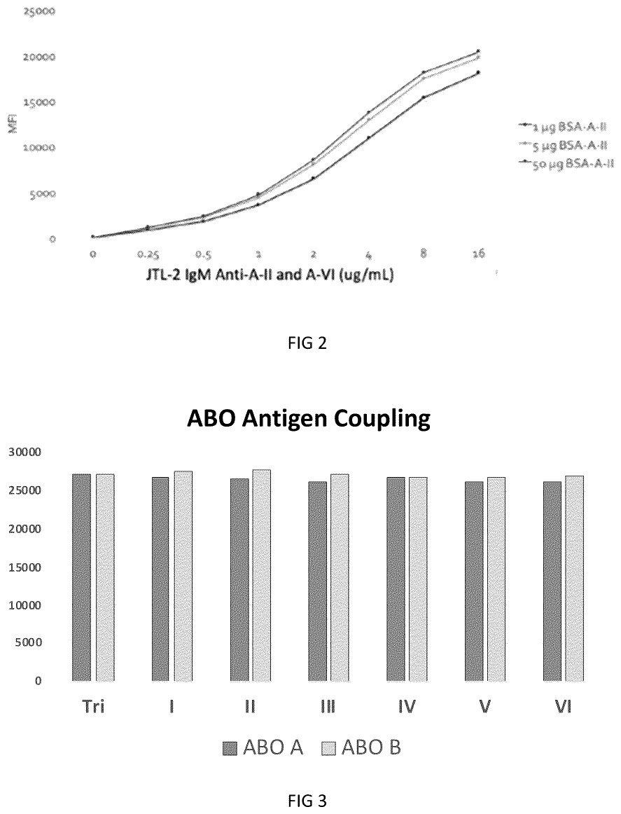

The optimum antigen coupling concentration was determined by titration using total amounts of 1 μg, 5 μg and 50 μg of antigen and relevant monoclonal antibody concentrations up to 16 μg/ml. As shown in FIG. 2 the optimum concentration of the BSA-conjugated glycans for bead coupling was determined to be about 5 μg/ml (containing 1.0×10⁶ beads/ml).

Additional beads were coupled to galactose-α-1,3-galactose (α-Gal) and to BSA as positive and negative control beads, respectively (Dextra, UK) using the same procedure as set out above. Blood group A- and β-trisaccharides were also coupled to individual beads (Dextra UK).

### Bead-Antigen Coupling Confirmation

Coupling of antigens to beads was confirmed using a panel of monoclonal antibodies either pan-reactive to ABO-A and ABO-B glycan structures or with specificities to individual ABO-A and -B glycan-subtype structures. The amount of antigen coupled to beads was consistent and comparable bead-to-bead as shown in FIG. 3. Additional details, including the specific monoclonal antibodies employed, are provided in Coupling and Coupling Confirmation standard operating protocols (SOPs) in Appendix A.

### Micro-Array Antibody Analysis

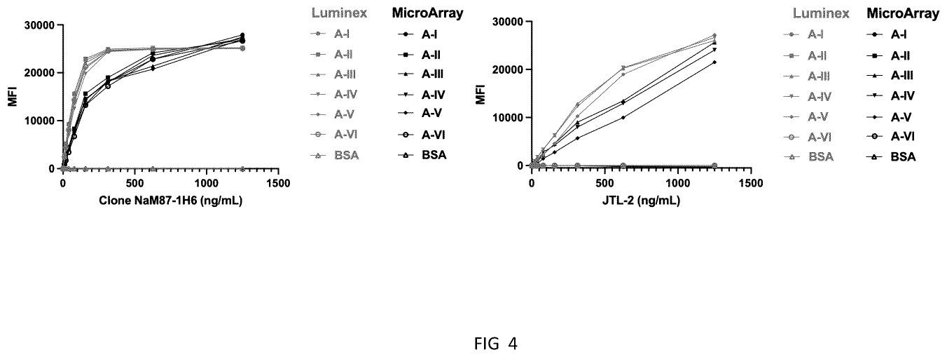

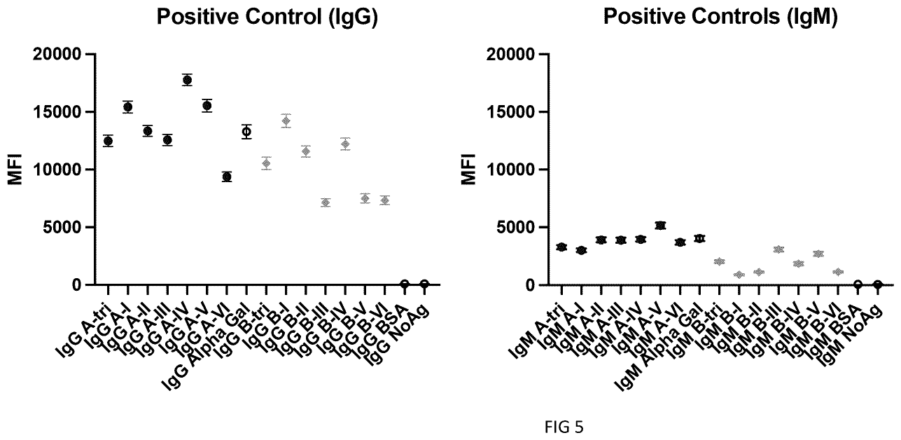

The monoclonal antibodies used in the bead coupling confirmation protocol were also run in parallel on the ABH-glycan microarray assay as previously described (Jeyakanthan et al. 2015, 2016). Antigens bound to glass arrays slides were identical to those coupled to the Luminex beads. The Luminex assay detected antibodies at low concentrations with greater sensitivity as compared to the glycan array (FIG. 4).

### Application of Assay Using Healthy Control Sera

Using antigen-coupled beads, profiles of anti-A and anti-B antibodies (and related antibodies) were characterized in serum or plasma samples from healthy adult individuals (n=143; ABO-O: n=68; ABO-A: n=48; ABO-B: n=17; ABO-AB: n=10) as described in the Luminex ABO Antibody Detection SOP (Appendix B). Briefly, 50 μL of diluted serum or plasma was incubated with pooled single antigen beads then incubated at room temperature with gentle agitation. The beads were washed three times prior to addition of either anti-human IgG or IgM secondary antibody. Secondary antibody concentrations were also optimized for this method. Plates were incubated again at room temperature with gentle agitation and washed three more times. The beads were resuspended in 80 μl of buffer and acquired on a Luminex® 200 instrument or a FlexMap 3D® Luminex instrument. When acquired on the FlexMap 3D®, the mean fluorescence intensity (MFI) results were divided by 1.67 to achieve MFI comparability between instruments. Each run included a positive and negative control serum.

The results of the assay were highly reproducible as shown in FIG. 5 demonstrating the results of the positive control sample tested over 24 individual runs.

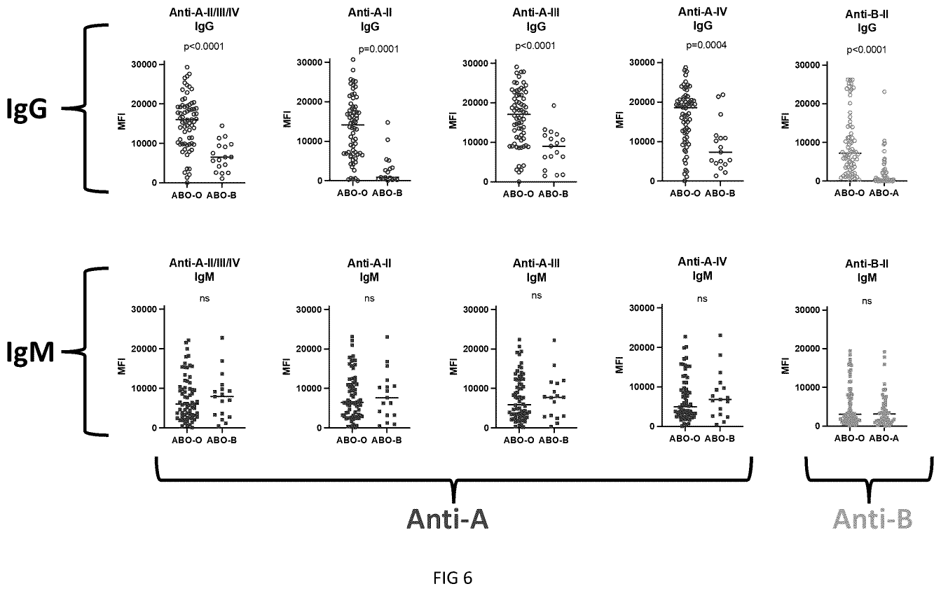

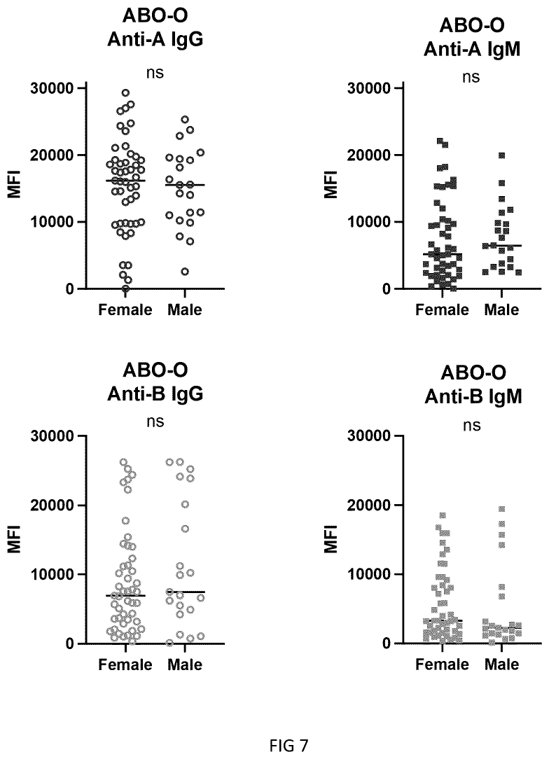

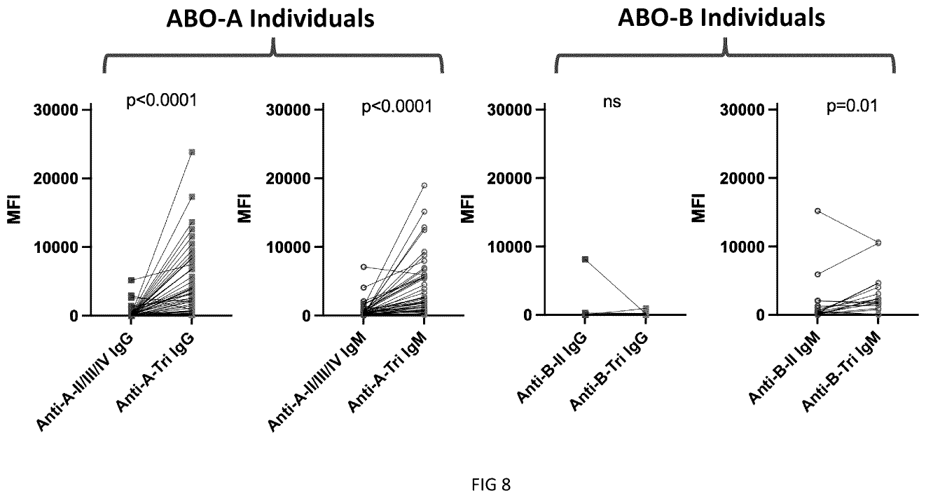

A comparison of IgG and IgM antibodies by blood group is shown in FIG. 6. Here and throughout the results, antibody analysis was focused on antibodies specific to A-II, -III, and -IV and B-II glycan subtypes as these are reported to be the most biologically relevant glycan targets. (Bentall et al. 2021; Clausen and Hakomori 1989; Jeyakanthan et al. 2016) Levels of antibodies with specificities to these A- and β-subtypes were highly variable between individuals. Additionally, sera from ABO-O individuals contained significantly higher levels of IgG isotype antibodies to A-II, -III, -IV and to B-II glycans than sera from ABO-B and ABO-A individuals, respectively, whereas there was no difference in levels of IgM isotype antibodies amongst sera from individuals of the different ABO blood groups. There were no significant sex-related differences observed (FIG. 7).

Antibodies to A and B trisaccharide targets were compared to antibodies with specificities for the cell and tissue relevant tetrasaccharide glycan subtypes A-II, -III, and -IV and B-II. Over half of the ABO-A individuals had detectable IgG and IgM antibodies to A-trisaccharide whereas less than 10% of the ABO-A blood group healthy controls exhibited reactivity to A-tetrasaccharide target beads (FIG. 8). This same finding was not observed for β-trisaccharide glycan vs B-II subtype glycan. This finding confirms that the trisaccharide targets are not an acceptable surrogate for the tetrasaccharide glycan subtype antigens.

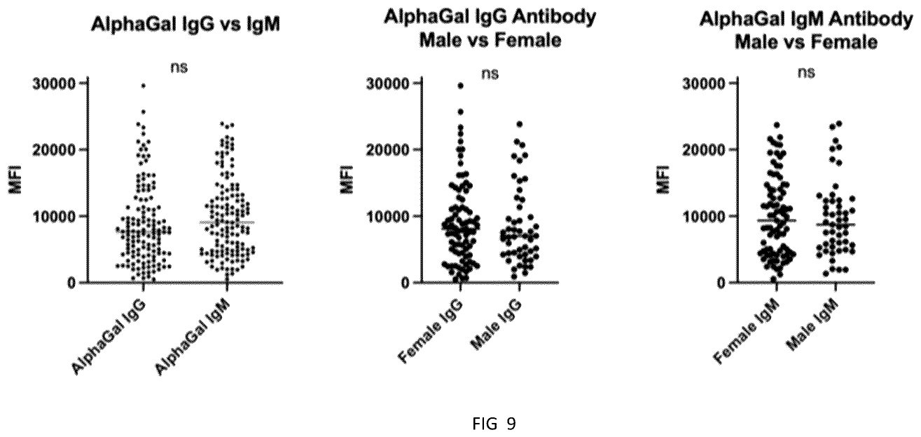

Similar levels of α-Gal antibodies of IgG vs IgM isotypes were detected and this observation remained true when analysing α-Gal antibodies by sex as shown in FIG. 9.

### Hemagglutination ABO Antibody Titre Testing

Traditional ABO titre testing was performed on individuals also tested by the present Luminex ABO antibody assay. Sufficient sample for agglutination testing was available for 119 of the 143 healthy controls. (ABO-O n=54, ABO-A, n=43, ABO-B n=16, ABO=AB, n=6). Serially diluted sera/plasma (50 μL) were incubated with 25 μL of 1% ABO-A1 and ABO-B reagent red cells (Referencells®, Immucor) at room temperature in a 96-well tray. The plate was mixed and allowed to incubate for one hour. Agglutination was read on an ELISPOT™ reader (CTL). The agglutination titre was reported as the last dilution showing visual agglutination. These agglutination scores were compared to the levels of tetrasaccharide-specific antibodies detected in the present Luminex assay.

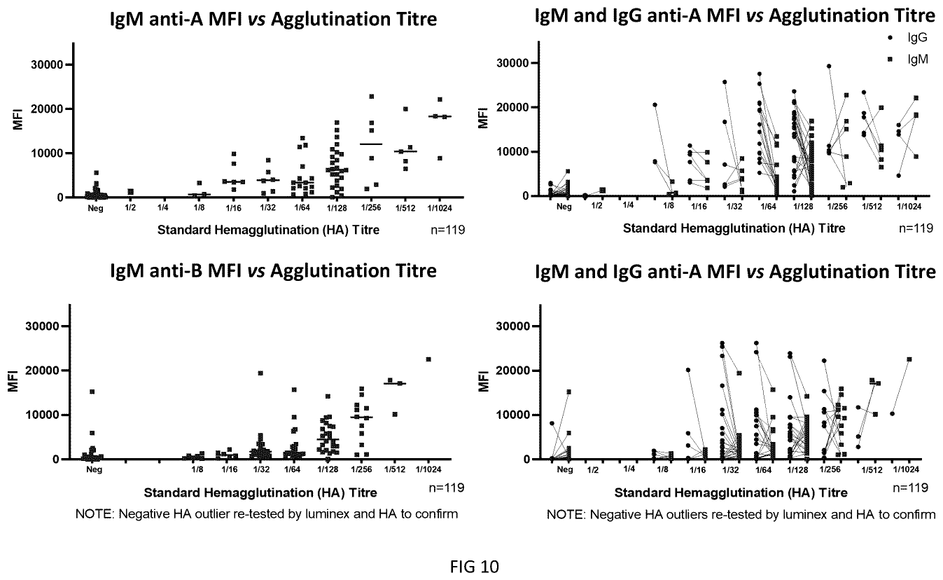

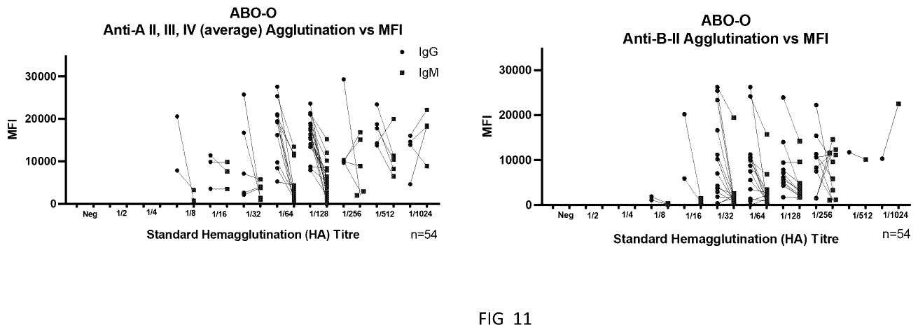

Within each anti-A and anti-B ABO hemagglutination titre, there was a high degree of variability of IgG and IgM antibodies detected by the present Luminex assay. There were increasing overall levels of IgM antibodies in each titre as shown in the IgM alone data in FIG. 10, but the levels of IgG and IgM antibodies in each titre are overlapping. There are two cases in which anti-A and anti-B tetrasaccharide antibodies were detected when a negative antibody titre had been reported; these results were repeated to confirm. Figure demonstrates this same comparison for ABO-O individuals only. Thus, not only is it impossible to determine the relevant subtype specificity in hemagglutinin assays, each titre demonstrates high variability of antibody levels as well as overlap of antibody levels across many titre results.

Application of Assay to Subjects with the Bombay Phenotype

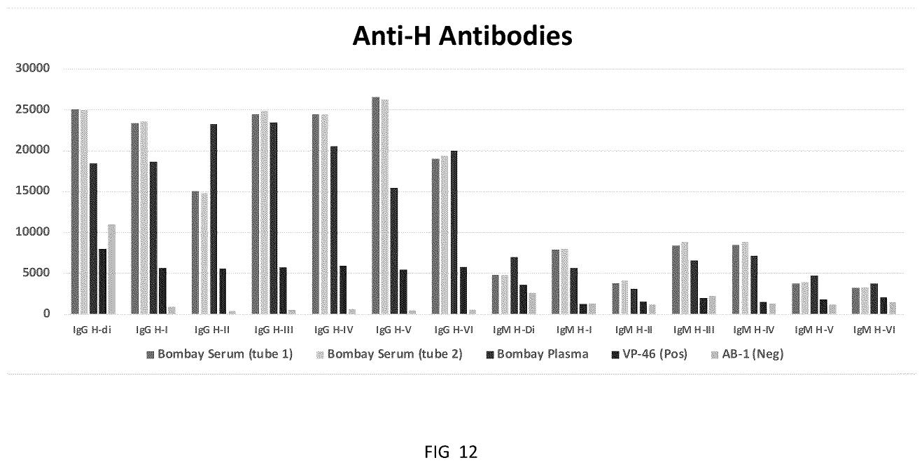

Using H antigen-coupled beads, profiles of anti-H antibodies (IgG and IgM) were characterized, using the present Luminex®-based assay, in serum or plasma samples from subjects with the Bombay phenotype. The results are illustrated in FIG. 12.

### Application of Assay to Renal Transplant Candidates

Using H antigen-coupled beads, profiles of anti-H antibodies in renal transplant candidates were characterized, using the present Luminex®-based assay, in serum or plasma samples from renal transplant candidates being assessed for ABO-A incompatible transplantation. These results illustrate the value of this assay in assessing xenotransplant recipients since xenotransplantation panels using this technology can include glycan targets to A, B, and H, and other antigens relevant to xenotransplantation, such as Neu5Gc, and Neu5Ac, as well as the positive control bead carrying α-Gal and the negative control bead. Additional glycans relevant to xenotransplantation can be readily incorporated into this assay by conjugating the glycan antigen(s) to a separate subset (or subsets) of bead for inclusion in the assay composition.

### Antibody Analysis Statistical Methods

All data were tested for normality using the Shapiro-Wilk, Anderson-Darling, and Kolmogorov-Smirnov tests. Non-parametric analysis of Mann-Whitney test and Wilcoxon test were used for paired and non-paired data, respectively when results were not normally distributed. The only normally distributed data were the positive control comparisons shown in FIG. 5. GraphPad Prism 9.3.1 was used for analysis and data graphing.

### DISCUSSION

The present Example describes the use of an embodiment of the multiplex assay of the present application to measure serum antibodies with specificities for ABO-A and -B glycans and demonstrate its reproducibility and utility in a study of samples from healthy adults. It has been widely reported that there is a need for better methods for the detection of ABO antibodies. (Denomme and Anani 2020). The field of histocompatibility has used a similar tool for HLA antibody detection for almost two decades. However, until the development of the present multiplex assay, attempts at a multiplex ABO evaluation/detection assay have been unsuccessful.

Clinical laboratories currently lack a reproducible assay for detection and precise characterisation of glycan subtype-specific IgG and IgM antibodies to support ABOi transplantation and transfusion. It is widely acknowledged that red cell agglutination methods are poorly standardized and that inter-laboratory comparisons of titre data are challenging (Bentall et al. 2016; Daga et al. 2021; Denomme and Anani 2020; JP, J, and LJ 2008; Kang et al. 2014). As demonstrated herein, the present Luminex®-based assay is highly reproducible and allows for accurate characterization of isotype and subtype-specificity of anti-A and anti-B antibodies, thus overcoming these barriers in clinical ABO antibody assessment.

The ease of differentiating the isotype of ABO antibodies also creates the potential to understand the risk of IgG vs IgM ABO antibodies in clinical ABO-incompatible (ABOi) organ and cell transplantation. While some studies have employed the use of dithiothreitol (DTT) and the addition of anti-human globulin (AHG) in an attempt to distinguish IgG from IgM ABO antibodies, this practice is inconsistent. While DTT is known to disassociate the IgM pentamer, it is not specific for IgM antibodies alone. There is also inconsistency in AHG titration methods, with some laboratories including DTT treatment and others not performing this treatment prior to ABO antibody titre testing. (Kahlyar et al. 2022)

It is widely recognized that there are discrepancies in organ transplant waitlist times based on ABO blood group of the patient. There is inequity in access to solid organ transplantation as blood group O and B patients wait longer to be transplanted with an ABO-compatible donor compared to A and AB patients. There is also an increasing need to consider ABOi transplantation as transplant registries globally are seeing increased numbers of patients with high levels of HLA antibodies resulting in poor access to HLA-compatible organs. (Hussey, Parameshwar, and Banner 2007) The success of ABOi transplantation suggests that disadvantaged blood groups ABO-O and ABO-B as well as HLA-sensitised patients would benefit from access to ABOi donors (Fan et al. 2004; de Weerd and Betjes 2018; West et al. 2001). However, the well-recognized limitations of red cell agglutination titre testing are a barrier for consistent and reproducible ABO antibody assessment within and between programs, and thus for accurate risk assessment, as well as for national transplant registry use.

Increased knowledge of subtype-specific antibodies in the setting of ABOi transplantation will facilitate advancements in other areas of transplant clinical practice and research. Improved histopathology studies of ABOi grafts could be carried out as antibodies to endothelial vs epithelial cells can be readily characterized. The pathology of transplant rejection is constantly evolving and this additional knowledge could be applied to retrospective and prospective studies. (Bruneval et al. 2017; Haas et al. 2017; Kobashigawa et al. 2018) Immune memory has been identified as an as a critical gap in transplant risk assessment but one that can be challenging to study (Tambur et al. 2018). The present Luminex®-based assay can be readily incorporated into immune memory assays for simultaneous assessment of HLA and ABO B cell memory (Karahan et al. 2018).

While the field of ABOi transplantation would clearly benefit from the use of this assay, there are also transfusion medicine applications. There is a growing demand for whole blood transfusion from ABO-O donors but naturally occurring anti-A and anti-B antibodies may cause transfusion reactions. (Yazer and Spinella 2018) Currently female whole blood donors are excluded due to reports that females have more ABO antibodies than males, however, the result of the present study did not show sex-based difference in the study population. The ABO blood group barrier is often crossed with platelet transfusions and IVIG, but transfusion medicine laboratories lack a consistent approach to measuring ABO antibodies in these blood products.

The ABO glycobiology field is hampered by lack of readily available monoclonal antibodies with specificity for individual glycan subytypes. The present Luminex®-based assay allows for clear characterization of A and B glycan monoclonal antibodies as shown in the coupling confirmation data from this assay development. This bead (microsphere) panel has been expanded to include H disaccharide and tetrasaccharide targets. H panel beads have been manufactured (data not shown) and can be included with the A and B beads for simultaneous antibody determination. The specificity of commonly used monoclonal antibodies in clinical transfusion medicine is also not well defined and this Luminex®-based assay can be used for this purpose.

It is possible that there are in-vivo glycan modifications to A and B tetrasaccharides that are not represented in the bead composition used in the current Example. However, if additional relevant glycans are determined to be biologically relevant, this composition can be easily expanded to include such targets using the same technology applied here.

The present Example did not assess the secretor status of the subjects in this study. Subtype I glycans are reported to be secreted in individuals with the corresponding fucosylsyl transferase 2 (FUT2) genotype. A-I and B-I subtype specific antibody patterns were observed that suggest the FUT2 non-secretor genotype in some individuals (data not shown). Secretor status is not known to be relevant in transplantation or routine transfusion practice but the present Luminex®-based assay can be used to study this further.

Further, the H panel mentioned above can be used to evaluate A, B, and H antibody profiles in individuals with Bombay phenotype who lack the normal fucosyltransferase 1 (FUT1) gene. This is illustrated in FIG. 12, which shows the ability of the present Luminex®-based assay to identify pan-reactive H reactivity in subjects having the Bombay phenotype.

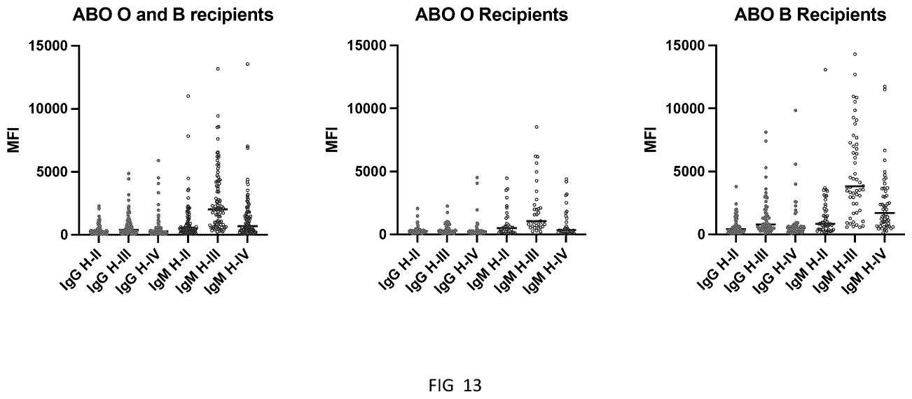

Transplantation has recently observed major steps forward in the field of xenotransplantation. (Cowan and Tector 2017; Montgomery et al. 2022; Porrett et al. 2022) This Luminex®-based assay can be incorporated in the recipient risk assessment algorithms as ABO-O swine may have different ABO-H and related glycans decorating their cells and tissues as compared to humans. (Milland and Sandrin 2006) Although the xenografts used in these recent transplants were from α-Gal knockout animals, there are other potential carbohydrate targets of interest and the present reproducible Luminex®-based assay can be expanded to include other xeno-glycans of interest, such as, but not limited to, Neu5Gc. FIG. 13 illustrates the use of the present assay in xenotransplantation for assessment of the presence of anti-H antibody in renal transplant candidates.

### CONCLUSION

In summary, this Example demonstrates that the present Luminex®-based assay is a highly reproducible ABO antibody detection assay that can be readily incorporated into clinical histocompatibility laboratories. The data presented herein demonstrate a high degree of variability in ABO antibody profiles and the inadequacy of red cell titration assays to report the complexity of these antibodies; detection of organ/tissue irrelevant antibodies by hemagglutination will also unnecessarily exclude individuals from access to ABO-incompatible transplantation. The present Luminex®-based assay can characterize A and B glycan subtype-specific antibodies in human serum and define antibody isotypes, overcoming the many limitations of the hemagglutination assay. This glycan-specific antibody detection assay can be used for allograft-specific determination of ABO antibody and immune risk evaluation in the setting of ABOi transplantation as well as transfusion and has the potential to significantly advance these fields. This method also lends itself readily to basic glycobiology research in multi-centre studies.

### REFERENCES

Angeloni, Stephan, Robert Cordes, Sherry Dunbar, Carlos Garcia, Grant Gibson, Charles Martin, and Valerie Stone. 2016. XMAP Cookbook: A Collection of Methods and Protocols for Developing Multiplex Assays with XMAP Technology. 3rd ed. Austin, TX: Luminex Corporation.
Bentall, Andrew, Mylvaganam Jeyakanthan, Manjit Braitch, Christopher W. Cairo, Todd L. Lowary, Stephanie Maier, Anne Halpin, Bruce Motyka, Lu Zou, Lori J. West, and Simon Ball. 2021. “Characterization of ABH-Subtype Donor-Specific Antibodies in ABO-A-Incompatible Kidney Transplantation.” American Journal of Transplantation 21 (11): 3649-62.
Clausen, H. and S. Hakomori. 1989. “ABH and Related Histo-Blood Group Antigens; Immunochemical Differences in Carrier Isotypes and Their Distribution.” Vox Sanguinis 56 (1): 1-20.
Clausen, H., S. B. Levery, E. Nudelman, S. Tsuchiya, and S. Hakomori. 1985. “Repetitive A Epitope (Type 3 Chain A) Defined by Blood Group A1-Specific Monoclonal Antibody TH-1: Chemical Basis of Qualitative A1 and A2 Distinction.” Proceedings of the National Academy of Sciences of the United States of America 82 (4): 1199-1203.
Daga, Sunil, Shimon Hussain, Nithya Krishnan, David Lowe, Manjit Braitch, Prashanth Patel, Andrew Bentall, Simon Ball, Daniel A. Mitchell, Robert Higgins, lan Skidmore, Daniel Zehnder, and David Briggs. 2021. “Immunoglobulin Isotype Compositions of ABO Specific Antibodies Are Dependent on the Individual Patient Blood Group and Blood Group Specificity: Results from a Healthy Donor Cohort.” Journal of Immunological Methods 494:113053.
Datta, S. S., S. Basu, M. Reddy, K. Gupta, and S. Sinha. 2021. “Comparative Evaluation of the Conventional Tube Test and Column Agglutination Technology for ABO Antibody Titration in Healthy Individuals: A Report from India.” Immunohematology 37 (1): 25-32.
Denomme, Gregory A. and Waseem Q. Anani. 2020. “ABO Titers: Harmonization and Identifying Clinically Relevant ABO Antibodies.” Transfusion 60 (3): 441-43.
El-Awar, N., J. Lee, and P. I. Terasaki. 2005. “HLA Antibody Identification with Single Antigen Beads Compared to Conventional Methods.” Human Immunology 66 (9): 989-97.
Jeyakanthan, M., P. J. Meloncelli, L. Zou, T. L. Lowary, I. Larsen, S. Maier, K. Tao, J. Rusch, R. Chinnock, N. Shaw, M. Burch, K. Beddows, L. Addonizio, W. Zuckerman, E. Pahl, J. Rutledge, K. R. Kanter, C. W. Cairo, J. M. Buriak, D. Ross, I. Rebeyka, and L. J. West. 2016. “ABH-Glycan Microarray Characterizes ABO Subtype Antibodies: Fine Specificity of Immune Tolerance After ABO-Incompatible Transplantation.” American Journal of Transplantation 16 (5): 1548-58.
Jeyakanthan, M., K. Tao, L. Zou, P. J. Meloncelli, T. L. Lowary, K. Suzuki, D. Boland, I. Larsen, M. Burch, N. Shaw, K. Beddows, L. Addonizio, W. Zuckerman, B. Afzali, D. H. Kim, M. Mengel, A. M. J. Shapiro, and L. J. West. 2015. “Chemical Basis for Qualitative and Quantitative Differences Between ABO Blood Groups and Subgroups: Implications for Organ Transplantation.” American Journal of Transplantation (10): 2602.
Kang, Seon Joo, Young Ae Lim, and Sae Yun Baik. 2014. “Comparison of ABO Antibody Titers on the Basis of the Antibody Detection Method Used.” Annals Of Laboratory Medicine 34 (4): 300-306.
Landsteiner, K. 2001. “[Agglutination phenomena of normal human blood].” Wiener klinische Wochenschrift 113 (20-21): 768-69.
Lane, William. 2016. “Next Generation Sequencing for Blood Group Antigen Profiling.” Blood 128 (22): SCI-24 LP-SCI-24.
de Mattos, Luiz Carlos. 2016. “Structural Diversity and Biological Importance of ABO, H, Lewis and Secretor Histo-Blood Group Carbohydrates.” Revista Brasileira de Hematologia e Hemoterapia 38 (4): 331-40.
Meloncelli, Peter J. and Todd L. Lowary. 2009. “Synthesis of ABO Histo-Blood Group Type V and VI Antigens *.” Australian Journal of Chemistry 62 (6): 558-74.
Meloncelli, Peter J. and Todd L. Lowary. 2010. “Synthesis of ABO Histo-Blood Group Type I and II Antigens.” Carbohydrate Research 345 (16): 2305-22.
Meloncelli, Peter J., Lori J. West, and Todd L. Lowary. 2011. “Synthesis and NMR Studies on the ABO Histo-Blood Group Antigens: Synthesis of Type Ill and IV Structures and NMR Characterization of Type I-VI Antigens.” Carbohydrate Research 346 (12): 1406-26.
Muthana, Saddam M. and Jeffrey C. Gildersleeve. 2016. “Factors Affecting Anti-Glycan IgG and IgM Repertoires in Human Serum.” Scientific Reports 6 (1): 19509.
Oriol, R., R. Mollicone, P. Coullin, A. M. Dalix, and J. J. Candelier. 1992. “Genetic Regulation of the Expression of ABH and Lewis Antigens in Tissues.” APMIS. Supplementum 27:28-38.
Pendu, J. Le, A. M. Dalix, R. Mollicone, K. Crainic, and R. Oriol. 1989. “EXPRESSION OF ABH, LEWIS AND RELATED TISSUE ANTIGENS IN THE HUMAN THYMUS.” International Journal of Immunogenetics 16 (1): 19-32.
Pochechueva, Tatiana, Alexander Chinarev, Marianne Spengler, Elena Korchagina, Viola Heinzelmann-Schwarz, Nicolai Bovin, and Robert Rieben. 2011. “Multiplex Suspension Array for Human Anti-Carbohydrate Antibody Profiling.” Analyst 136 (3): 560-69.
Ravn, V. and E. Dabelsteen. 2000. “Tissue Distribution of Histo-Blood Group Antigens.” APMIS: Acta Pathologica, Microbiologica, et Immunologica Scandinavica 108 (1): 1-28.
Stussi, G., K. Huggel, H. U. Lutz, U. Schanz, R. Rieben, and J. D. Seebach. 2005. “Isotype-Specific Detection of ABO Blood Group Antibodies Using a Novel Flow Cytometric Method.” British Journal of Haematology 130 (6): 954-63.
Sullivan, Harold C., Howard M. Gebel, and Robert A. Bray. 2017. “Understanding Solid-Phase HLA Antibody Assays and the Value of MFI.” Human Immunology.
Svensson, L., L. Rydberg, L. C. De Mattos, and S. M. Henry. 2009. “Blood Group A1 and A2 Revisited: An Immunochemical Analysis.” Vox Sanguinis 96 (1): 56-61.
Tait, Brian D., Caner Susal, Howard M. Gebel, Peter W. Nickerson, Andrea A. Zachary, Frans
H. J. Claas, Elaine F. Reed, Robert A. Bray, Patricia Campbell, Jeremy R. Chapman, P. Toby Coates, Robert B. Colvin, Emanuele Cozzi, Ilias I. N. Doxiadis, Susan V Fuggle, John Gill, Denis Glotz, Nils Lachmann, Thalachallour Mohanakumar, Nicole Suciu-Foca, Suchitra Sumitran-Holgersson, Kazunari Tanabe, Craig J. Taylor, Dolly B. Tyan, Angela Webster, Adriana Zeevi, and Gerhard Opelz. 2013. “Consensus Guidelines on the Testing and Clinical Management Issues Associated with HLA and Non-HLA Antibodies in Transplantation.” Transplantation 95 (1): 19-47.

### Appendix A

### Standard Operating Procedure: Coupling Confirmation of Luminex® Beads

Purpose: This SOP outlines the procedure for the assessment of coupling success/efficiency of the A or B glycan subtypes, and controls (BSA and aGal) to Luminex beads.¹ This step follows the coupling and must be performed before the beads are used. It is to be run in parallel with the most recent lot/batch of beads. This coupling confirmation must be followed with another QC run of human negative and positive control as well as other recently tested sera as further QC for the new beads.

This protocol can also be used for quality control of beads that have been in storage or perform shipment/lot QC on new Novoclone™ and secondary antibody shipments. Note that the specific concentration of Novoclone™ reagents is not provided by the vendor and that the vendor QC for this reagent is performed by hemagglutination. As such, there may be some variation from lot to lot on this Luminex® platform which makes lot QC on the Luminex® beads very important for the Novoclone™ reagents.

### Materials:

| Reagents | Supplies | Equipment |
|---|---|---|
| A/B glycan-coupled beads | 96-well plate Corning | Luminex cytometer |
| BSA-coupled beads | 3605 (round bottom | Vortex |
| αGal glycan-coupled beads | non-binding white | Plate shaker |
| PBS-TBN buffer | polystyrene) | (Barnstead Labline) |
| Novaclone anti-A | Adhesive plastic plate | Luminex Magnetic |
| Immucor (cat# 5170022) | seal | Plate Separator |
| Novaclone anti-B | 1.5 mL micro- | (Part# CN-0269-01) |
| Immucor (cat# 517502) | centrifuge tubes | Pipettes |
| JTL-2² | Disposable pipette | P10 |
| JTL-4² | tips | P200 |
| PE-labeled goat anti- | Aluminum foil | P1000 |
| mouse IgM secondary | | P200 multi |
| antibody Southern | | |
| Biotech (cat# 1020-09) | | |

Specimen: Specimens are not used at this stage of the Luminex® bead-based ABH antigen assay, as this SOP provides an outline for the coupling confirmation of ABO-BSA-Antigen coupled Luminex® beads to be used later to test specimens. Only monoclonal antibodies are required.
Safety: See specific SDS for detailed information.
Quality Control (QC): Previously characterized antibodies are used for this purpose. New lot # or new antibodies must be tested in parallel with previously confirmed coupled bead lot/batch (or in parallel with new reagent). A PBS-PBN well is included to ensure that there is no non-specific fluorescence to any specific bead.

Procedure:

| | | |
|---|---|---|
| Step | Detail | Information |

Confirm with laboratory personnel that the Luminex instrument will be
available; request that it is left on if it is already in use
Assemble required reagents as listed above, selecting the appropriate
antibody-coupled microsphere set(s) and ensure that plate shaker is available
Warm PBS-TBN to room temperature

| | | | | |
|---|---|---|---|---|
| 1. | Design tray layout | 1.1. | Retrieve an Excel Coupling Confirmation | Excel worksheet will |
| | and complete Excel | | Worksheet from the server drive | calculate required bead |
| | Coupling | 1.2. | Prepare tray layout to include all monoclonal | volumes to achieve 2500 |
| | Confirmation | | antibodies tested. For a full A/B panel this | beads per well |
| | Worksheet | | includes: | Measure extra bead volume |
| | | | Novoclone anti-A | for 2 extra wells |
| | | | Novoclone anti-B | Details on antibody |
| | | | JTL-2 | specificity and dilutions are |
| | | | JTL-2 | provided in the Additional |
| | | | PBS-TBN (background control) | Information Section of this |
| | | 1.3. | Fill in information, such as: | SOP |
| | | | Bead Counts | Refer to last worksheet to |
| | | | Date of Run | confirm the required dilutions |
| | | | Lot information of Novaclones | to test. |
| | | | Preparation date of the JTL reagents | |
| | | | Lot information of anti-mouse IgM | |
| | | | secondary | |
| | | | Required bead volume per well | |
| 2. | Prepare beads | 2.1. | Label a 1.5 mL microcentrifuge tube as | |
| | | | pooled beads and with the date | |
| | | 2.2. | Vortex each individual bead preparation for | |
| | | | 30 seconds | |
| | | 2.3. | Add the pre-determined bead volume for | |
| | | | each panel (A and B) into a microcentrifuge | |
| | | | tube as was calculated in step 1 | |
| | | 2.4. | Spin the tube in a swing-out microcentrifuge | |
| | | | at 8000 g for 2 minutes | |
| | | 2.5. | Remove the supernatant using a pipette | |
| | | 2.6. | Add the determined volume of PBS-TBN | |
| | | | from Excel worksheet | |
| 3. | Prepare Novaclone | 3.1. | Prepare dilutions of each of Novaclone anti- | Anti-A is blue |
| | dilutions | | A and Novaclone anti-B dilutions using PBS- | Anti-B is yellow |
| | | | TBN as the diluent and document the | The 1/100 dilution will |
| | | | preparation of the dilutions in the plate lay | provide saturated MFI values |
| | | | out tab of the worksheet | Note that the 1/1600 dilution |
| | | | 1/100 | will not result in a negative |
| | | | 1/200 | (low MFI) result and if |
| | | | 1/400 | titration to negative is |
| | | | 1/800 | required, dilution up to |
| | | | 1/1600 | approximately 1/5000 |
| | | 3.2. | Document the preparation of the dilutions in | |
| | | | the plate lay out tab of the worksheet | |
| 4. | Prepare the JLT 2 | 4.1. | Prepare JTL-2 and -4 dilutions using PBS- | These dilutions will titrate to |
| | and 4 dilutions | | TBN as the diluent | negative. If saturated bead |
| | | | 1250 ng/mL | dilutions are required, higher |
| | | | 625 ng/mL | concentrations can be made |
| | | | 312.5 ng/mL | from the stock solution |
| | | | 156.25 ng/mL | |
| | | | 78.13 ng/mL | |
| | | | 39.06 ng/mL | |
| | | | 19.53 ng/mL | |
| | | 4.2. | Document the preparation of the dilutions in | |
| | | | the plate lay out tab of the worksheet | |
| 5. | Add beads and | 5.1. | Refer to plate layout and mark the columns | Some coloured sharpie pens |
| | monoclonal antibody | | and rows to be used using a black sharpie | may affect fluorochromes and |
| | dilutions | 5.2. | Add 5 μL of the multiplexed (pooled) bead | black is preferable |
| | | | suspension to each well as per tray layout | |
| | | 5.3. | Add 50 μL of each Novaclone anti-A and B | |
| | | | dilution as per tray layout | |
| | | 5.4. | Add 50 μL of each JLT 2 and 4 dilution as | |
| | | | per tray layout | |
| | | 5.5. | Tap plate to mix | |
| 6. | Incubate the plate | 6.1. | Apply a fresh adhesive plate seal to the plate | This precaution is taken to |
| | fox 30 minutes at | | ensuring the edges of the outside wells are | protect the bead |
| | room temperature | | sealed | fluorochromes from light |
| | | 6.2. | Wrap the plate in aluminum foil | |
| | | 6.3. | Incubate the plate for 30 minutes at room | |
| | | | temperature on a plate shaker at setting ‘2’ | |

Perform step 7 during the step 6 incubation

| | | | | |
|---|---|---|---|---|
| 7. | Prepare secondary | 7.1. | Prepare the PE-labeled anti-mouse IgM | |
| | antibody | | secondary antibody at a 1/100 dilution | |
| | | 7.2. | Document the preparation of the secondary | |
| | | | antibody in the plate lay out tab of the | |
| | | | worksheet | |
| | | 7.3. | Protect the secondary antibody from light by | |
| | | | wrapping in foil or placing in a drawer | |
| 8. | Wash 3 times with | 8.1. | Carefully remove adhesive plate seal. | Avoid splashes or liquid |
| | PBS-TBN buffer | 8.2. | Add 150 uL of PBS-TBN buffer to each well | transfer from well to well |
| | | | using an 8 channel multipipette | |
| | | 8.3. | WITHOUT REMOVING THE PLATE | |
| | | | FROM THE MAGNET, flick the plate with | |
| | | | one firm motion | |
| | | | Do this over a non-handwashing sink or | |
| | | | biohazard reservoir. | |
| | | 8.4. | WITHOUT FLIPPING THE PLATE BACK | |
| | | | OVER, dab the plate onto a paper towel to | |
| | | | capture any residual droplets | |
| | | 8.5. | Perform the second and third washes by | |
| | | | adding 200 uL of wash buffer and repeating | |
| | | | steps 8.3 and 8.4 twice | |
| 9. | Add PE-labeled | 9.1. | Add 100 μL of the prepared 1/100 diluted | |
| | anti-mouse IgM | | PE-labeled anti-mouse IgM secondary | |
| | secondary antibody | | antibody to each well as per tray layout | |
| | | 9.2. | Re-seal the plate with a fresh adhesive seal | |
| | | 9.3. | Tap plate to mix | |
| 10. | Incubate the plate | 11.1. | Wrap plate in aluminum foil | This precaution is taken to |
| | for 30 minutes at | 11.2. | Incubate for 30 minutes at room temperature | protect the plate from light |
| | room temperature | | on a plate shaker at setting ‘2’ | |
| 11. | | | | |
| 12. | Wash microspheres | 12.1. | Remove adhesive plate seal. | |
| | 3 times and | 12.2. | Add 100 uL of PBS-TBN buffer to each | |
| | resuspend in PBS- | | well | |
| | TBN buffer | 12.3. | Place plate on a magnetic separator for 2 | |
| | | | minutes | |
| | | 12.4. | WITHOUT REMOVING THE PLATE | |
| | | | FROM THE MAGNET, flick the plate with | |
| | | | one firm motion | |
| | | | Do this over a non-handwashing sink or | |
| | | | biohazard reservoir. | |
| | | 12.5. | WITHOUT FLIPPING THE PLATE | |
| | | | BACK OVER, dab the plate onto a paper | |
| | | | towel to capture any residual droplets | |
| | | 12.6. | Perform the second and third washes by | |
| | | | adding 200 uL of wash buffer and repeating | |
| | | | steps 12.3 to 12.5 twice | |
| | | 12.7. | Resuspend in 80 uL of TBN-PBS wash | |
| | | | buffer | |
| 13. | Acquire the beads | 13.1. | Acquire beads on the Luminex flow | This step is to be done as soon |
| | | | cytometer | as possible and within two |
| | | | | hours |

Procedural Notes
Sterile conditions are not required for these steps.

### Calculating Beads Required

The Excel worksheet will calculate the beads as per the following calculations. 2500 beads are needed per well (with 2 extra wells included for pipetting loss of reagent)

### Beads Calculation

C₁V₁=2500 beads: Where C₁=concentration of beads in original stock solution of coupled beads (beads/μL); Where:
V₁=volume of beads to add to each well from the original stock solution (μL);

### Example Calculation

C₁V₁=2500 beads where: C₁=concentration of beads/mL (ex 3.75=10⁵);
V₁=x (#μL to be determined per well);
(3.75×10⁵ beads/mL)(x)=2500 beads
x=6.7 μL beads per well, then bring up to 50 μL

Secondary Antibody Preparation of 1/100—prepare in 3 mL Falcon tube (#wells+1)/100 μL=total volume required

Example calculation: (14+1)×100 μL=1500 μL
1/1000
1/1000=x/1500 μL
x=1.5 μL
1.5 82 L+1500 μL PBS-TBN

### Appendix B

### Luminex Human Sample ABO Antibody Detection

### Purpose

This SOP outlines the procedure for the measurement of IgG and IgM ABO subtype-specific antibodies using Lumex single antigen beads. This assay was adapted for Luminex from the microarray method published by Jeyakantham et al¹

This assay uses a Lainex bead-based panel of ABO-A and ABO-B subtype I-VI glycans which have been coupled to individual Laminex beads. Additional beads to bovine serum albumin (BSA), galactose-α-1,3-galactose (α-Gal) glycan, and A- and B-trisaccharides are also included in this panel.

Phycoerythrin (PE) secondary antibodies specific to human IgG and IgM are used to detect ARO antibodies bound to individual beads. Beads can be acquired on & Luminex 200™ or FlexMap 3D® Laminex instrument.

### Materials

| Reagents | Supplies | Equipment |
|---|---|---|
| ABO bead panel | 96-well plate, Corning #3605 | Microcentrifuge |
| Negative control serum | round bottom non-binding | Luminex Magnetic Plate |
| (AB-1) | white polystyrene, non- | Separator |
| Positive control serum (VP-5) | binding surface, 330 μL | (Part# CN-0269-01) |
| TBN-PBS wash buffer | capacity, 75-200 μL working | Vortex |
| IgM secondary antibody: | volume | Plate shaker |
| PE-Anti-Human IgM, Cat# | Plate seal, Excel Scientific | Pipettes (P10, P200, P1000) |
| IGM-PEC1, | Sealing Film 100SEALPLT) | and tips |
| One Lambda Thermo Fisher | 1.5 mL clear microfuge tubes, | 300 uL volume multi- |
| IgG secondary antibody: | MCT-150-C Axygen 311-08- | channel pipette (8 well) |
| PE-Anti-Human IgG, Cat# LS- | 051 | |
| AB2, | Disposable pipette tips | |
| One Lambda Thermo Fisher | | |

Specimen This method has been validated for use with serum, heparin, and EDTA plasma. Samples do not require EDTA testament
Safety Individual SDS for the beads and wash buffer are stored in the West laboratory. There are no safety concerns w the
Quality Control There are negative and positive control sera included in this essay
   AB 1: Plasma nuit from as ABO-AB individual source from the University of Alberta Transfusion Medicine Laboratory
   VP-5: Pool of two ABO-O plasma units sourced from the University of Alberta Transfusion Medicine Laboratory

### Control Beads Include:

BSA coupled bead (negative control/background bead)
No antigen bead (negative control/background bead)
α-Gal as a (positive control bead)
Precautions Universal precautions must be taken when handling human specimens

Procedure

| | | |
|---|---|---|
| Step | Detail | Information |

Turn on Luminex instrument and perform standard daily start up and calibration
Bring the wash buffer to room temperature

| | | | | |
|---|---|---|---|---|
| 1. | Initiate and | 1.1. | Open the ‘in use’ Excel template for this | Bead numbers vary from |
| | document samples | | bead batch | batch to batch of beads. |
| | in the excel ABO | 1.2. | Fill in the number of samples run to | Ensure the correct version for |
| | plate worksheet | | calculate the bead volumes required in the | this bead batch date is used |
| | | | ‘Beadcalc’ tab | The Excel worksheet will |
| | | 1.3. | Fill in the plate map details for each sample | automatically calculate the |
| | | | to be tested as well as the negative and | required bead volumes for |
| | | | positive control and assign locations on the | each bead address |
| | | | plate in the ‘LAYOUT’ tab | |
| | | 1.4. | Document the lot# of the secondary | |
| | | | antibody reagents in the ‘Reagent’ tab of the | |
| | | | worksheet | |
| 2. | Prepare the bead | 2.1. | Using the calculations in the Excel sheet | |
| | mix | | from Step 1, prepare the bead mix to be | |
| | | | used for all beads and controls | |
| 3. | Prepare | 3.1. | Centrifuge samples in microfuge at 10,000 | EDTA treatment is not |
| | samples/controls and | | rpm for 5 minutes | required |
| | perform dilutions | 3.2. | Using the TBN wash buffer, prepare 1/25 | Sample calculation: |
| | | | dilutions of all samples and the controls | 1/25 dilation for each sample: |
| | | | | 8 uL + 192 uL wash buffer |
| | | | | 1/50 dilution for pos control: |
| | | | | 4 uL + 196 uL wash buffer |
| 4. | Prepare plate adding | 4.1. | Add 5 μL ABO A and B beads to every well | As per plate layout tab of |
| | beads and samples | | as indicated on plate worksheet | Excel worksheet |
| | | 4.2. | Add 50 uL of each diluted sample and control | |
| | | | to the IgG as well as IgM wells on the plate | |
| | | | worksheet | |
| 5. | Seal and place on | 5.1. | Apply plate/tray seal | |
| | plate shaker for 30 | 5.2. | Wrap plate in aluminum foil | This precaution is taken to |
| | minutes | 5.3. | Incubate for 30 minutes at room | protect the plate from light |
| | | | temperature on the plate shaker at setting ‘2’ | |
| 6. | Prepare secondary | 6.1. | Prepare the IgG second antibody at a | 100 uL are needed for each |
| | antibodies | | 1/50 dilution | well. Prepare enough for one |
| | | 6.2. | Prepare the IgM secondary antibody at a | extra well |
| | | | 1/50 dilution | Example of calculations for a |
| | | | | run of 12 IgG wells: |
| | | | | 28 uL of IgG |
| | | | | secondary Ab |
| | | | | 1372 uL of wash |
| | | | | buffer |

These beads are the MagPlex beads so they can be ‘pelleted’ using a magnet, centrifuge or vacuum
apparatus. The following steps are using a magnet wash method.

| | | | | |
|---|---|---|---|---|
| 7. | Wash plate 3 times | 7.1. | Remove plate/tray seal | If an alternate wash method is |
| | | 7.2. | Using the 8 channel multi-pipette, add | used, document on the plate |
| | | | 150 uL of wash buffer to each well | layout worksheet |
| | | 7.3. | Place the plate on the Luminex Magnetic | Ensure that face cover is used |
| | | | Plate separator and fold up the side clips to | to prevent exposure to |
| | | | secure the plate | aerosols |
| | | 7.4. | Leave the plate on the magnet for 2 minutes | |
| | | 7.5. | WITHOUT REMOVING THE PLATE | |
| | | | FROM THE MAGNET, flick the plate with | |
| | | | one firm motion | |
| | | | Do this over a non-handwashing sink or | |
| | | | biohazard reservoir. | |
| | | 7.6. | WITHOUT FLIPPING THE PLATE | |
| | | | BACK OVER, dab the plate onto a paper | |
| | | | towel to capture any residual droplets | |
| | | 7.7. | Perform the second and third washes by | |
| | | | adding 200 uL of wash buffer and repeating | |
| | | | steps 7.3 to 7.5 twice | |
| 8. | Add the IgG and | 8.1. | To the designated IgG wells, add 100 μL of | Refer to plate map in |
| | IgM secondary | | the prepared 1/50 PE labeled IgG secondary | worksheet for well locations |
| | antibodies | | antibody | |
| | | 8.2. | To the designated IgM wells, add 100 μL of | |
| | | | the prepared 1/50 PE labeled IgG secondary | |
| | | | antibody | |
| | | 8.3. | Apply a plate/tray seal | |
| | | 8.4. | Tap plate to gently mix | |
| 9. | Place on plate | 9.1. | Wrap plate in aluminum foil | This precaution is taken to |
| | shaker for 30 | 9.2. | Incubate for 30 minutes at room | protect the plate from light |
| | minutes | | temperature on the plate shaker at setting ‘2’ | |
| 10. | Wash plate 3 times | 10.1. | Remove plate/tray seal | |
| | | 10.2. | Place the plate on the Luminex Magnetic | |
| | | | Plate separator and fold up the side clips to | |
| | | | secure the plate | |
| | | 10.3. | Leave the plate on the magnet for 2 minutes | |
| | | 10.4. | WITHOUT REMOVING THE PLATE | |
| | | | FROM THE MAGNET, flick the plate with | |
| | | | one firm motion | |
| | | | Do this over a non-handwashing sink or | |
| | | | biohazard reservoir. | |
| | | 10.5. | WITHOUT FLIPPING THE PLATE | |
| | | | BACK OVER, dab the plate onto a paper | |
| | | | towel to capture any residual droplets | |
| | | 10.6. | Perform the second and third washes by | |
| | | | adding 200 uL of wash buffer and repeating | |
| | | | steps 10.3 to 10.5 twice | |
| 11. | Resuspend beads | 11.1. | Add 80 μL of wash buffer to each well and | Please acquire beads within 2 |
| | for acquisition | | gently mix | hours. If a delay is necessary, |
| | | | | please document on the Tray |
| | | | | Worksheet |
| 12. | Acquire the beads | 12.1. | Read/acquire on the Luminex cytometer | This step is to be done as |
| | | | | soon as possible and within |
| | | | | two hours |

Procedural Notes
Sterile conditions are not required for this SOP.
The optimal dilution for serum/plasma samples as well as PE-labelled anti-human IgG and IgM was determined by performing a checkerboard experiment

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A method of identifying an ABO histo-blood subtype antibody profile of a subject comprising:
(a) incubating a biological sample obtained from said subject with a bead composition, wherein the bead composition comprises:
(i) a plurality of subsets of Luminex® beads where each of the subsets within the plurality are individually coupled to a Type I, Type II, Type III, Type IV ABO-A or ABO-B subtype glycan antigen;
(ii) a first control subset of Luminex® beads that are coupled to a negative control antigen and function as a negative control;
(iii) a second control subset of Luminex® beads that are not coupled to any antigen and function as a further negative control; and
(iv) a third control subset Luminex® beads that are coupled to a positive control antigen and function as a positive control;
(b) washing the incubated mixture from step (a);
(c) incubating the washed mixture from step (b) with secondary anti-IgG and anti-IgM antibodies, wherein the secondary antibodies are labeled with a fluorescent label;
(d) washing the incubated mixture from step (c) to remove unbound secondary antibodies;
(e) measuring fluorescence associated with the subsets of Luminex® beads; and
(f) generating the ABO antigen subtype antibody profile of the subject from the fluorescence data collected in step (e),
wherein the subsets of beads coupled to the ABO-A and ABO-B subtype glycans each comprise a bovine serum albumin (BSA)-linked ABO subtype antigen coupled to the corresponding beads at an amount of about 5 μg of the BSA-linked ABO subtype antigen per 1.0×10⁶ beads.

2. The method according to claim 1, wherein the BSA-linked Type I, Type II, Type III, Type IV ABO-A or ABO-B subtype glycan antigens are:
A Type I α-D-GalpNAc-(1→3)-[α-L-Fucp-(1→2)]-β-D-Galp-(1→3)-β-D-GlcpNAc-BSA,
A Type II α-D-GalpNAc-(1→3)-[α-L-Fucp-(1→2)]-β-D-Galp-(1→4)-β-D-GlcpNAc-BSA,
A Type III α-D-GalpNAc-(1→3)-[α-L-Fucp-(1→2)]-β-D-Galp-(1→3)-α-D-GalpNAc-BSA,
A Type IV α-D-GalpNAc-(1→3)-[α-L-Fucp-(1→2)]-β-D-Galp-(1→3)-β-D-GalpNAc-BSA,
B Type I α-D-Galp-(1→3)-[α-L-Fucp-(1→2)]-β-D-Galp-(1→3)-β-D-GlcpNAc-BSA,
B Type II α-D-Galp-(1→3)-[α-L-Fucp-(1→2)]-β-D-Galp-(1→4)-β-D-GlcpNAc-BSA,
B Type III α-D-Galp-(1→3)-[α-L-Fucp-(1→2)]-β-D-Galp-(1→3)-α-D-GalpNAc-BSA, and
B Type IV α-D-Galp-(1→3)-[α-L-Fucp-(1→2)]-β-D-Galp-(1→3)-β-D-GalpNAc-BSA.

3. The method according to claim 1, wherein the bead composition additionally comprises two subsets of Luminex® beads coupled to BSA-linked Type V ABO-A and ABO-B subtype glycan antigens at an amount of about 5 μg of the BSA-linked Type V ABO subtype antigen per 1.0×10⁶ beads.

4. The method according to claim 3, wherein the BSA linked Type V ABO-A and ABO-B subtype glycan antigens are:
A Type V α-D-GalpNAc-(1→3)-[α-L-Fucp-(1→2)]-β-D-Galp-(1→3)-β-D-Galp-BSA, and
B Type Va-D-Galp-(1→3)-[α-L-Fucp-(1→2)]-β-D-Galp-(1→3)-β-D-Galp-BSA.

5. The method according to claim 1, wherein the bead composition additionally comprises two subsets of Luminex® beads coupled to BSA-linked Type VI ABO-A and ABO-B subtype glycan antigens at an amount of about 5 μg of the BSA-linked Type V ABO subtype antigen per 1.0×10⁶ beads.

6. The method according to claim 5, wherein the BSA linked Type VI ABO-A and ABO-B subtype glycan antigens are:
A Type VI α-D-GalpNAc-(1→3)-[α-L-Fucp-(1→2)]-β-D-Galp-(1→4)-β-D-Glcp-BSA, and
B Type VI α-D-Galp-(1→3)-[α-L-Fucp-(1→2)]-β-D-Galp-(1→4)-β-D-Glcp-BSA.

7. The method according to claim 1, wherein the bead composition additionally comprises subsets of Luminex® beads individually coupled to BSA-linked Type I, Type II, Type III, Type IV, Type V and Type VI ABO-H subtype glycan antigens at an amount of about 5 μg of the BSA-linked ABO-H subtype antigen per 1.0×10⁶ beads.

8. The method according to claim 7, wherein the BSA-linked Type I, Type II, Type III, Type IV, Type V and Type VI ABO-H subtype glycan antigens are:
H Type I α-L-Fucp-(1→2)-β-D-Galp-(1→3)-β-D-GlcpNAc-BSA,
H Type II α-L-Fucp-(1→2)-β-D-Galp-(1→4)-β-D-GlcpNAc-BSA,
H Type III α-L-Fucp-(1→2)-β-D-Galp-(1→3)-α-D-GalpNAc-BSA,
H Type IV α-L-Fucp-(1→2)-β-D-Galp-(1→3)-β-D-GalpNAc-BSA,
H Type V α-L-Fucp-(1→2)-β-D-Galp-(1→3)-β-D-Galp-BSA, and
H Type VI α-L-Fucp-(1→2)]-β-D-Galp-(1→4)-β-D-Glcp-BSA.

9. The method according to claim 1, wherein the bead composition additionally comprises one or more subsets of Luminex® beads individually coupled to BSA-linked antigen(s) present in a donor animal for a xenotransplant, wherein the antigen(s) present in the donor animal is, optionally, N-Glycolylneuraminic acid (Neu5Gc), N-acetylneuraminic acid (Neu5Ac), or both.

10. The method according to claim 1, wherein the positive control antigen is galactose-α-1,3-galactose (α-Gal) and/or wherein the negative control is bovine serum albumin (BSA).

11. (canceled)

12. A kit for identifying an ABO histo-blood subtype antibody profile of a subject comprising:
(a) a bead composition, wherein the bead composition comprises:
(i) a plurality of subsets of Luminex® beads where each of the subsets within the plurality are individually coupled to a Type I, Type II, Type III, Type IV ABO-A or ABO-B subtype glycan antigen;
(ii) a first control subset of Luminex® beads that are coupled to a negative control antigen and function as a negative control;
(iii) a second control subset of Luminex® beads that are not coupled to any antigen and function as a further negative control; and
(iv) a third control subset Luminex® beads that are coupled to a positive control antigen and function as a positive; and
(b) instructions for use of the kit to perform the method of claim 1.

13. The kit according to claim 12, wherein the BSA-linked Type I, Type II, Type III, Type IV ABO-A or ABO-B subtype glycan antigens are:
A Type I α-D-GalpNAc-(1→3)-[α-L-Fucp-(1→2)]-β-D-Galp-(1→3)-β-D-GlcpNAc-BSA,
A Type II α-D-GalpNAc-(1→3)-[α-L-Fucp-(1→2)]-β-D-Galp-(1→4)-β-D-GlcpNAc-BSA,
A Type III α-D-GalpNAc-(1→3)-[α-L-Fucp-(1→2)]-β-D-Galp-(1→3)-α-D-GalpNAc-BSA,
A Type IV α-D-GalpNAc-(1→3)-[α-L-Fucp-(1→2)]-β-D-Galp-(1→3)-β-D-GalpNAc-BSA,
B Type I α-D-Galp-(1→3)-[α-L-Fucp-(1→2)]-β-D-Galp-(1→3)-β-D-GlcpNAc-BSA,
B Type II α-D-Galp-(1→3)-[α-L-Fucp-(1→2)]-β-D-Galp-(1→4)-β-D-GlcpNAc-BSA,
B Type III α-D-Galp-(1→3)-[α-L-Fucp-(1→2)]-β-D-Galp-(1→3)-α-D-GalpNAc-BSA, and
B Type IV α-D-Galp-(1→3)-[α-L-Fucp-(1→2)]-β-D-Galp-(1→3)-β-D-GalpNAc-BSA.

14. The kit according to claim 12, wherein the bead composition additionally comprises two subsets of Luminex® beads coupled to BSA-linked Type V ABO-A and ABO-B subtype glycan antigens at an amount of about 5 μg of the BSA-linked Type V ABO subtype antigen per 1.0×10⁶ beads.

15. The kit according to claim 14, wherein the BSA linked Type V ABO-A and ABO-B subtype glycan antigens are:
A Type V α-D-GalpNAc-(1→3)-[α-L-Fucp-(1→2)]-β-D-Galp-(1→3)-β-D-Galp-BSA, and
B Type V α-D-Galp-(1→3)-[α-L-Fucp-(1→2)]-β-D-Galp-(1→3)-β-D-Galp-BSA.

16. The kit according to claim 12, wherein the bead composition additionally comprises two subsets of Luminex® beads coupled to BSA-linked Type VI ABO-A and ABO-B subtype glycan antigens at an amount of about 5 μg of the BSA-linked Type V ABO subtype antigen per 1.0× 10⁶ beads.

17. The kit according to claim 16, wherein the BSA linked Type VI ABO-A and ABO-B subtype glycan antigens are:
A Type VI α-D-GalpNAc-(1→3)-[α-L-Fucp-(1→2)]-β-D-Galp-(1→4)-β-D-Glcp-BSA, and
B Type VI α-D-Galp-(1→3)-[α-L-Fucp-(1→2)]-β-D-Galp-(1→4)-β-D-Glcp-BSA.

18. The kit according to claim 12, wherein the bead composition additionally comprises subsets of Luminex® beads individually coupled to BSA-linked Type I, Type II, Type III, Type IV, Type V and Type VI ABO-H subtype glycan antigens at an amount of about 5 μg of the BSA-linked ABO-H subtype antigen per 1.0×10⁶ beads.

19. The kit according to claim 18, wherein the BSA-linked Type I, Type II, Type III, Type IV, Type V and Type VI ABO-H subtype glycan antigens are:
H Type I α-L-Fucp-(1→2)-β-D-Galp-(1→3)-β-D-GlcpNAc-BSA,
H Type II α-L-Fucp-(1→2)-β-D-Galp-(1→4)-β-D-GlcpNAc-BSA,
H Type III α-L-Fucp-(1→2)-β-D-Galp-(1→3)-α-D-GalpNAc-BSA,
H Type IV α-L-Fucp-(1→2)-β-D-Galp-(1→3)-β-D-GalpNAc-BSA,
H Type V α-L-Fucp-(1→2)-β-D-Galp-(1→3)-β-D-Galp-BSA, and
H Type VI α-L-Fucp-(1→2)]-β-D-Galp-(1→4)-β-D-Glcp-BSA.

20. The kit according to claim 12, wherein the bead composition additionally comprises one or more subsets of Luminex® beads individually coupled to BSA-linked antigen(s) present in a donor animal for a xenotransplant, wherein the antigen(s) present in the donor animal is, optionally, N-Glycolylneuraminic acid (Neu5Gc), N-acetylneuraminic acid (Neu5Ac), or both.

21. The kit according to claim 12, wherein the positive control antigen is galactose-α-1,3-galactose (α-Gal) and/or wherein the negative control antigen is bovine serum albumin (BSA).

22. (canceled)
